SEQUENCE LISTING (26 sequences)

SEQ ID NO: 1 - DNA, 2427 bp, Mus musculus
atgcgtgacaagggccggcgccaggcagtgcgtggcccggccttcatgttcggtgctcgtgggcccagcctcacagctgaggaggagcgcttcctggatgctgcggagtacggcaacatccctgtggtgcgcaagatgctggaggagtctcgcacgctcaatgtcaactgcgtggactacatgggccagaacgcactgcagctggctgtaggcaatgagcatctggaggtgaccgagctgctgctgaagaaggagaacctggcgcggatcggtgatgcgctgctgctagccatcagcaagggctatgtacgcatcgtggaggccatcctgggccacccaggctttgcagccagccggcgcctgaccctcagcccttgcgagcaagaactgcgagatgatgacttctatgcctatgacgaggatggcacacgcttctcgcctgacatcacgcccatcatcctggctgcacactgccataagtacgaggtggtgcacctgctgctactcaagggtgcacgcatcgagaggccacacgactacttctgtcgctgctctgactgcgctgagaaacaaaggcttgacgccttcagccactcaaggtctaggatcaatgcctacaagggactggccagcccagcatacttgtcgctgtccagcgaggaccccgtgctcacagcactggaactcagcaatgagctggccaagctggccaacatagagaaggagttcaagaatgactacaggaaactctccatgcaatgcaaagacttcgtagtgggtgtgctggacctgtgtcgggactcggaggaggtggaagccattctgaatggagatctggaatcagccgagcccctggaaagacacgggcacaaagcgtcactgagtcgtgtcaaacttgccattaagtatgaagtcaaaaagtttgtggctcaccccaactgccaacagcagcttttgacgatctggtatgagaacctctccggcctccgggagcagaccatcgctatcaagtgtctggtcgtgttggtcgtggccttgggtcttccattcctcgccattggctattggattgcaccttgtagcaggctggggaagattcttcgaagccccttcatgaagttcgtggcccacgctgcctccttcatcatctttctgggtctgctcgtattcaacgcctcggacaggtttgaaggcatcaccacgctgccaaacatcactgttatcgactaccccaagcaaatcttcagggtgaagaccacccagtttacatggacggaaatgttaattatggtctgggttcttgggatgatgtggtctgagtgcaaggagctgtggctggaggggccccgggagtacatcgtgcagttgtggaatgtgcttgatttcggcatgctctccatcttcatcgccgcgttcacagccaggttcctcgctttcctgcaggccacgaaggcgcagcagtatgtggatagtcacgtgcaggagagcgatctgagcgaagtcacgctcccacctgaggttcaatatttcacctatgctagagacaaatggctcccctcagaccctcagatcatatctgaagggctctatgccatagctgtggtgctcagcttctcccggattgcgtacatcctccctgcaaacgagagctttgggcccttgcagatctctcttggaaggactgtgaaggacatattcaagttcatggttctcttcattatggtgttcctggctttcatgattggcatgttcatactttattcttactaccttggggccaaagtaaatcctgcttttaccacggttgaagaaagtttcaagactttgttttggtccatatttggactgtctgaagtgacttctgttgtcctcaaatatgatcacaaattcatagagaatattggctatgttctttatgggatatataatgtaactatggtggtcgttttactcaacatgctaattgctatgattaatagctcataccaagagatcgaggatgacagtgatgtagaatggaagtttgctcgttccaaactttggctctcctactttgatgatggaaaaacattacctccacccttcagtctggtccctagtccaaagtcatttgtttacttcatcatgaggatcactaacttttccaaatgcaggaggagaagactgcagaaggatctggaactgggcatgggtaactcaaagtccaggcagataatgaaaagactcataaaacggtatgttttgaaagcacaagtagacaaagaaaacgatgaggtgaatgaaggtgaactgaaagaaatcaagcaggatatctccagccttcgttatgaactcttagaagataagagccaagcgacggaggaattagccatcttgattcataaactcagtgagaaactgaaccccagtgtgctgagatgtgagtga

SEQ ID NO: 2 - protein, 808 aa, Mus musculus
MRDKGRRQAVRGPAFMFGARGPSLTAEEERFLDAAEYGNIPVVRKMLEESRTLNVNCVDYMGQNALQLAVGNEHLEVTELLLKKENLARIGDALLLAISKGYVRIVEAILGHPGFAASRRLTLSPCEQELRDDDFYAYDEDGTRFSPDITPIILAAHCHKYEVVHLLLLKGARIERPHDYFCRCSDCAEKQRLDAFSHSRSRINAYKGLASPAYLSLSSEDPVLTALELSNELAKLANIEKEFKNDYRKLSMQCKDFVVGVLDLCRDSEEVEAILNGDLESAEPLERHGHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVIDYPKQIFRVKTTQFTWTEMLIMVWVLGMMWSECKELWLEGPREYIVQLWNVLDFGMLSIFIAAFTARFLAFLQATKAQQYVDSHVQESDLSEVTLPPEVQYFTYARDKWLPSDPQIISEGLYAIAVVLSFSRIAYILPANESFGPLQISLGRTVKDIFKFMVLFIMVFLAFMIGMFILYSYYLGAKVNPAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRITNFSKCRRRRLQKDLELGMGNSKSRQIMKRLIKRYVLKAQVDKENDEVNEGELKEIKQDISSLRYELLEDKSQATEELAILIHKLSEKLNPSVLRCE

SEQ ID NO: 3 - DNA, 2511 bp, Mus musculus
atgcgtgacaagggccggcgccaggcagtgcgtggcccggccttcatgttcggtgctcgtgggcccagcctcacagctgaggaggagcgcttcctggatgctgcggagtacggcaacatccctgtggtgcgcaagatgctggaggagtctcgcacgctcaatgtcaactgcgtggactacatgggccagaacgcactgcagctggctgtaggcaatgagcatctggaggtgaccgagctgctgctgaagaaggagaacctggcgcggatcggtgatgcgctgctgctagccatcagcaagggctatgtgcgcatcgtggaggccatcctgggccacccaggctttgcagccagccggcgcctgaccctcagcccttgcgagcaagaactgcgagatgatgacttctatgcttacgacgaggatggcacacgcttctcgcctgacatcacgcccatcatcctggctgcacactgccataagtacgaggtggtgcacctgctgctactcaagggtgcacgcatcgagaggccacacgactacttctgtcgctgctctgactgcgctgagaaacaaaggcttgacgccttcagccactcaaggtctaggatcaatgcctacaagggactggccagcccagcatacttgtcgctgtccagcgaggaccccgtgctcacagcactggagctcagcaatgagctggccaagctggccaacatagagaaggagttcaagaatgactacaggaaactctccatgcaatgcaaagacttcgtagtgggtgtgctggacctgtgtcgggactcggaggaggtggaagccattctgaatggagatctggaatcagccgagcccctggaaagacacgggcacaaagcgtcactgagtcgtgtcaaacttgccattaagtatgaagtcaaaaagtttgtggctcaccccaactgccaacagcagcttttgacaatctggtatgagaacctttccggcctccgggagcagaccatcgctatcaagtgtctggtcgtgttggtcgtggccttgggtcttccattcctcgccatcggctattggattgcaccttgtagcaggctggggaagattcttcgaagccccttcatgaagtttgtggcccacgctgcctccttcatcatctttctgggtctgcttgtattcaacgcctcggacagatttgaaggcatcaccacgctgccaaacatcactgttatcgactaccccaagcaaatcttcagggtgaagacaacccagttcacgtggacggaaatgttaattatggtctgggttcttgggatgatgtggtctgagtgcaaggagctgtggctggaggggcccggggagtacatcgtgcagttgtggaatgtgcttgatttcggcatgctctccatcttcatcgccgcgttcacagccaggttcctcgctttcctgcaggccacgaaggcgcagcagtatgtggatagtcacgtgcaggagagcgatctgagcgaagtcacgctcccacctgaggttcaatatttcacctatgctagagacaaatggctcccctcagaccctcagatcatatctgaagggctctatgccatagctgtggtgctcagcttctcccggattgcgtacatcctccctgcaaacgagagctttgggcccttgcagatctctcttggaaggactgtgaaggacatattcaagttcatggttctcttcattatggtgttcctggctttcatgattggcatgttcatactttattcttactaccttggggccaaagtaaatcctgcttttaccacggttgaagaaagtttcaagactttgttttggtccatatttggactgtctgaagtgacttctgttgtcctcaaatatgatcacaaattcatagagaatattggctatgttctttatgggatatataatgtaactatggtggtcgttttactcaacatgctaattgctatgattaatagctcataccaagagatcgaggatgacagtgatgtagaatggaagtttgctcgttccaaactttggctctcctacttcgatgatggaaaaacattacctccacccttcagtctggtccctagtccaaaatcatttgtttacttcatcatgaggatcactaacttttccaaatgcaggaggagaagactgcagaaggatctggaactgggcatgggtaactcaaagtccaggttaaacctcttcacacagtctaactcgagagtttttgaatcacacagttttaacagcattctcaatcagccaacacgatatcagcagataatgaaaagactcataaaacggtatgttttgaaagcacaagtagacaaagaaaacgatgaggtgaatgaaggtgaactgaaagaaatcaagcaggatatctccagccttcgttatgaacttttagaagataagagccaagcgacggaggaattagccatcttgattcataaactcagtgagaaactgaaccccagtgtgctgagatgtgagtga

SEQ ID NO: 4 - protein, 836 aa, Mus musculus
MRDKGRRQAVRGPAFMFGARGPSLTAEEERFLDAAEYGNIPVVRKMLEESRTLNVNCVDYMGQNALQLAVGNEHLEVTELLLKKENLARIGDALLLAISKGYVRIVEAILGHPGFAASRRLTLSPCEQELRDDDFYAYDEDGTRFSPDITPIILAAHCHKYEVVHLLLLKGARIERPHDYFCRCSDCAEKQRLDAFSHSRSRINAYKGLASPAYLSLSSEDPVLTALELSNELAKLANIEKEFKNDYRKLSMQCKDFVVGVLDLCRDSEEVEAILNGDLESAEPLERHGHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVIDYPKQIFRVKTTQFTWTEMLIMVWVLGMMWSECKELWLEGPGEYIVQLWNVLDFGMLSIFIAAFTARFLAFLQATKAQQYVDSHVQESDLSEVTLPPEVQYFTYARDKWLPSDPQIISEGLYAIAVVLSFSRIAYILPANESFGPLQISLGRTVKDIFKFMVLFIMVFLAFMIGMFILYSYYLGAKVNPAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRITNFSKCRRRRLQKDLELGMGNSKSRLNLFTQSNSRVFESHSFNSILNQPTRYQQIMKRLIKRYVLKAQVDKENDEVNEGELKEIKQDISSLRYELLEDKSQATEELAILIHKLSEKLNPSVLRCE

SEQ ID NO: 5 - DNA, 2433 bp, Rattus norvegicus
atggcgaggccgagggagccgagccgcagcgccgccgccggggctggaggggcgtcaacggcgggctggagccgccctgcccgcgcgcgccgccgtccccggggcccgacgcagtacggcaacatccctgtggtgcgcaagatgctggaggagtctcgcacgctcaatgtcaactgcgtggactacatgggccagaatgcgctgcagctggccgtgggcaatgagcatctggaggtgaccgagctgctgctgaagaaggagaacctggcgcgtatcggtgatgcgctgctgctcgccatcagcaaaggctatgtgcgcatcgtggaggccatcctaggccacccaggctttgcggccagccggcgcctgaccctcagcccttgcgagcaagaactgcgcgatgacgacttctatgcttacgacgaggatggcacacgcttctcacctgacatcacgcccatcatcctggctgcgcattgccataaatacgaggtggtgcacctgctgctactcaagggtgcacgcatcgagcggccacacgactacttctgtcgctgcgccgactgtgctgagaagcaaaggcttgatgccttcagccactcaaggtctaggatcaatgcctacaagggactggccagcccagcatacttgtcgctgtccagcgaggaccctgtgctcacagcgctggaactcagcaatgagctggccaagctggccaacatagagaaggagttcaagaatgactacaggaagctctccatgcagtgcaaagacttcgtagtaggtgtgctggacctgtgccgggactcagaggaggtggaagccattctgaatggagatctggaatcggtggaacccctggagagacacgggcacaaggcgtcgctgagtcgggtcaaacttgccattaaatatgaagtcaaaaagtttgtggctcaccccaactgccaacagcagcttttgaccatctggtacgagaacctctcgggccttcgggagcagaccatcgctatcaagtgtctggtcgtgttggtcgtggccttgggccttccattcctcgccatcggctactggattgcaccttgtagcaggctggggaaaattcttcgaagccccttcatgaagttcgtggctcacgctgcctccttcatcatcttcctgggtctgcttgtgttcaacgcctcagaccggtttgaaggcatcaccacgctgcccaacatcaccgttattgactaccccaagcaaatcttcagggtgaagaccacccagttcacatggacagaaatgctaattatggtctgggttctcgggatgatgtggtctgagtgcaaggagctgtggctggaggggccccgggagtacatcgtgcagctgtggaacgtgcttgacttcgggatgctctccatcttcattgctgccttcaccgccaggttcctagcgtttctgcaagccaccaaagcgcagcagtatgtggacagccacgtgcaggagagcgacctgagcgaagtcacactcccaccagaggttcagtatttcacctatgctagagataaatggcttccttctgaccctcagatcatatcggaaggcctctatgccatagctgtggtgctcagcttctcccggatcgcgtacattctccctgcaaacgagagctttgggcccttgcagatctctctggggaggactgtgaaggacatattcaagttcatggttctcttcatcatggtgttcctggctttcatgattggcatgttcatactttactcctactaccttggggccaaagtaaaccctgcttttaccacggttgaagaaagtttcaagactttgttttggtccatatttggactctctgaagtgacttctgttgtcctcaaatatgaccacaaattcatagagaacattggctatgtcctttatggaatatacaatgtaactatggtggtcgttctgctcaacatgctgattgctatgattaacagctcataccaagaaatcgaggatgacagtgatgtagagtggaagtttgctcgttccaaactctggctatcctacttcgatgatggaaaaacattacctccacccttcagtctggtccctagtccaaaatcgtttgtttatttcatcatgaggatcactaacttttccaaatgcaggaggagaagacttcagaaggatctggaactgggcatgggtaactcaaagtccaggcagataatgaaaagactcataaaacggtatgttttgaaagcacaagtagacaaagaaaatgatgaggtgaacgaaggtgaactgaaagaaatcaagcaggatatctccagccttcgttacgaacttttggaagataagagccaagcgacggaggaactggccatcttgattcataaactcagtgagaaactgaaccccagtgcgctgaggtgtgaatga

SEQ ID NO: 6 - protein, 810 aa, Rattus norvegicus
MARPREPSRSAAAGAGGASTAGWSRPARARRRPRGPTQYGNIPVVRKMLEESRTLNVNCVDYMGQNALQLAVGNEHLEVTELLLKKENLARIGDALLLAISKGYVRIVEAILGHPGFAASRRLTLSPCEQELRDDDFYAYDEDGTRFSPDITPIILAAHCHKYEVVHLLLLKGARIERPHDYFCRCADCAEKQRLDAFSHSRSRINAYKGLASPAYLSLSSEDPVLTALELSNELAKLANIEKEFKNDYRKLSMQCKDFVVGVLDLCRDSEEVEAILNGDLESVEPLERHGHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVIDYPKQIFRVKTTQFTWTEMLIMVWVLGMMWSECKELWLEGPREYIVQLWNVLDFGMLSIFIAAFTARFLAFLQATKAQQYVDSHVQESDLSEVTLPPEVQYFTYARDKWLPSDPQIISEGLYAIAVVLSFSRIAYILPANESFGPLQISLGRTVKDIFKFMVLFIMVFLAFMIGMFILYSYYLGAKVNPAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRITNFSKCRRRRLQKDLELGMGNSKSRQIMKRLIKRYVLKAQVDKENDEVNEGELKEIKQDISSLRYELLEDKSQATEELAILIHKLSEKLNPSALRCE

SEQ ID NO: 7 - DNA, 2517 bp, Rattus norvegicus
atggcgaggccgagggagccgagccgcagcgccgccgccggggctggaggggcgtcaacggcgggctggagccgccctgcccgcgcgcgccgccgtccccggggcccgacgcagtacggcaacatccctgtggtgcgcaagatgctggaggagtctcgcacgctcaatgtcaactgcgtggactacatgggccagaatgcgctgcagctggccgtgggcaatgagcatctggaggtgaccgagctgctgctgaagaaggagaacctggcgcgtatcggtgatgcgctgctgctcgccatcagcaaaggctatgtgcgcatcgtggaggccatcctaggccacccaggctttgcggccagccggcgcctgaccctcagcccttgcgagcaagaactgcgcgatgacgacttctatgcttacgacgaggatggcacacgcttctcacctgacatcacgcccatcatcctggctgcgcattgccataaatacgaggtggtgcacctgctgctactcaagggtgcacgcatcgagcggccacacgactacttctgtcgctgcgccgactgtgctgagaagcaaaggcttgatgccttcagccactcaaggtctaggatcaatgcctacaagggactggccagcccagcatacttgtcgctgtccagcgaggaccctgtgctcacagcgctggaactcagcaatgagctggccaagctggccaacatagagaaggagttcaagaatgactacaggaagctctccatgcagtgcaaagacttcgtagtaggtgtgctggacctgtgccgggactcagaggaggtggaagccattctgaatggagatctggaatcggtggaacccctggagagacacgggcacaaggcgtcgctgagtcgggtcaaacttgccattaaatatgaagtcaaaaagtttgtggctcaccccaactgccaacagcagcttttgaccatctggtacgagaacctctcgggccttcgggagcagaccatcgctatcaagtgtctggtcgtgttggtcgtggccttgggccttccattcctcgccatcggctactggattgcaccttgtagcaggctggggaaaattcttcgaagccccttcatgaagttcgtggctcacgctgcctccttcatcatcttcctgggtctgcttgtgttcaacgcctcagaccggtttgaaggcatcaccacgctgcccaacatcaccgttattgactaccccaagcaaatcttcagggtgaagaccacccagttcacatggacagaaatgctaattatggtctgggttctcgggatgatgtggtctgagtgcaaggagctgtggctggaggggccccgggagtacatcgtgcagctgtggaacgtgcttgacttcgggatgctctccatcttcattgctgccttcaccgccaggttcctagcgtttctgcaagccaccaaagcgcagcagtatgtggacagccacgtgcaggagagcgacctgagcgaagtcacactcccaccagaggttcagtatttcacctatgctagagataaatggcttccttctgaccctcagatcatatcggaaggcctctatgccatagctgtggtgctcagcttctcccggatcgcgtacattctccctgcaaacgagagctttgggcccttgcagatctctctggggaggactgtgaaggacatattcaagttcatggttctcttcatcatggtgttcctggctttcatgattggcatgttcatactttactcctactaccttggggccaaagtaaaccctgcttttaccacggttgaagaaagtttcaagactttgttttggtccatatttggactctctgaagtgacttctgttgtcctcaaatatgaccacaaattcatagagaacattggctatgtcctttatggaatatacaatgtaactatggtggtcgttctgctcaacatgctgattgctatgattaacagctcataccaagaaatcgaggatgacagtgatgtagagtggaagtttgctcgttccaaactctggctatcctacttcgatgatggaaaaacattacctccacccttcagtctggtccctagtccaaaatcgtttgtttatttcatcatgaggatcactaacttttccaaatgcaggaggagaagacttcagaaggatctggaactgggcatgggtaactcaaagtccaggttaaacctcttcactcagtctaactcgagagtttttgaatcacacagttttaacagcattctcaatcagccaacacgatatcagcagataatgaaaagactcataaaacggtatgttttgaaagcacaagtagacaaagaaaatgatgaggtgaacgaaggtgaactgaaagaaatcaagcaggatatctccagccttcgttacgaacttttggaagataagagccaagcgacggaggaactggccatcttgattcataaactcagtgagaaactgaaccccagtgcgctgaggtgtgaatga

SEQ ID NO: 8 - protein, 838 aa, Rattus norvegicus
MARPREPSRSAAAGAGGASTAGWSRPARARRRPRGPTQYGNIPVVRKMLEESRTLNVNCVDYMGQNALQLAVGNEHLEVTELLLKKENLARIGDALLLAISKGYVRIVEAILGHPGFAASRRLTLSPCEQELRDDDFYAYDEDGTRFSPDITPIILAAHCHKYEVVHLLLLKGARIERPHDYFCRCADCAEKQRLDAFSHSRSRINAYKGLASPAYLSLSSEDPVLTALELSNELAKLANIEKEFKNDYRKLSMQCKDFVVGVLDLCRDSEEVEAILNGDLESVEPLERHGHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVIDYPKQIFRVKTTQFTWTEMLIMVWVLGMMWSECKELWLEGPREYIVQLWNVLDFGMLSIFIAAFTARFLAFLQATKAQQYVDSHVQESDLSEVTLPPEVQYFTYARDKWLPSDPQIISEGLYAIAVVLSFSRIAYILPANESFGPLQISLGRTVKDIFKFMVLFIMVFLAFMIGMFILYSYYLGAKVNPAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRITNFSKCRRRRLQKDLELGMGNSKSRLNLFTQSNSRVFESHSFNSILNQPTRYQQIMKRLIKRYVLKAQVDKENDEVNEGELKEIKQDISSLRYELLEDKSQATEELAILIHKLSEKLNPSALRCE

SEQ ID NO: 9 - DNA, 2427 bp, Cavia porcellus
atgcgggagaagggccggcgccaggcggtccggggtccggccttcatgttcaacgaccgcggcaccagcctcacgccggaggaggagcgcttcctcgacgccgccgagtacggcaacatcccggtggtgcgcaagatgctggaggagtcccggacgctgaacgtcaactgcgtggactacatgggccagaacgcgctgcagctggccgtgggcaacgagcacctggaggtcaccgagctgctgctcaagaaggagaacctggcgcgcatcggcgacgcgctgctgctggccatcagcaagggctacgtgcgcatcgtggaggccatcctcaaccaccccggcttcgcggccagccggcgcctcaccctcagcccctgcgagcaggagctgcaggacgacgacttctacgcctacgacgaggacggcacgcgcttctcgcccgacatcacgcccatcatcctggccgcgcactgccagaagtacgaggtggtgcacatgctgctgctgaagggcgccaggatcgagcggccgcacgactacttctgcaagtgcgcggactgcgccgagaagcagcgccacgactccttcagccactcgcgctccaggatcaacgcctacaaggggctggccagccccgcgtacctgtcgctgtccagcgaggacccggtgctcacggccctggagctcagcaacgagctggccaagctggccaacatcgagaaggagttcaagaacgactacagaaagctgtccatgcaatgcaaagactttgtagtgggtgtgctggatctttgccgggactcggaagaggtcgaagccattctgaatggagatctggaatccacagagcctctggaagtgcacaggcataaagcttcgctgagtcgtgtcaaacttgccatcaagtatgaagtaaagaagtttgtagctcaccccaactgccagcagcagcttttgacgatttggtacgaaaacctctcgggcctgcgggaacagactgtcgccatcaagtgtctggtggtgctggttgtggccttgggccttccgtttcttgccattggctattggatcgcaccttgcagcaggctggggaaaatcctgcgaagccctttcatgaagttcgtggcgcatgcagcttcgttcatcatcttcctgggcctgctcgtgttcaatgcctctgacaggttcgagggcattgccacgctgcccaacatcaccgtcatcgactaccccaagcagatcttccgggtgaaaaccactcagttcacctggacagaaatgctaatcatggtctgggttctgggaatgatgtggtctgagtgtaaggagctctggctggagggacctagagaatacattttgcagttgtggaatgtgcttgacttcgggatgctttccatcttcattgctgctttcacagccagattcctagccttccttcaagccacaaaagcccaacagtatgtggacagttatgtccaagagagcgacctcagtgaagtcactctcccgccagagatacagtatttcacttatgctagagataaatggctcccttctgacccccagatcatatctgaaggcctctacgccatagctgtcgtgctcagcttctctcggatcgcatacatcctccctgcaaacgagagcttcggccccttgcagatctccctgggaaggactgtgaaggacatattcaagttcatggtcctcttcattatggtgtttctggcctttatgattggcatgttcatactttattcttactaccttggggccaaagtaaactctgcttttaccactgtagaagaaagtttcaagactttgttttggtcaatatttgggttgtctgaagtgacttcagttgtgcttaaatatgatcacaaattcatagagaacattggatacgttctttatggaatatacaatgtaactatggtggtcgttttgctcaacatgctaattgctatgattaatagctcatatcaagaaattgaggatgacagtgatgtagaatggaagtttgcacgttcaaagctctggttatcctattttgatgatggaaagactttacctccacccttcagcctggttcccagcccaaaatcctttgtttatttcatcatgcggatcattaacttttccaaatgtagaaggagaaggctgcagaaagatctggaaatgggaatgggtaactcaaagtccaggcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccgttctgattcataaactgagtgagaaactgaatcccagcgtgctgagatgtgaatga

SEQ ID NO: 10 - protein, 808 aa, Cavia porcellus
MREKGRRQAVRGPAFMFNDRGTSLTPEEERFLDAAEYGNIPVVRKMLEESRTLNVNCVDYMGQNALQLAVGNEHLEVTELLLKKENLARIGDALLLAISKGYVRIVEAILNHPGFAASRRLTLSPCEQELQDDDFYAYDEDGTRFSPDITPIILAAHCQKYEVVHMLLLKGARIERPHDYFCKCADCAEKQRHDSFSHSRSRINAYKGLASPAYLSLSSEDPVLTALELSNELAKLANIEKEFKNDYRKLSMQCKDFVVGVLDLCRDSEEVEAILNGDLESTEPLEVHRHKASLSRVKLAIKYEVKKFVAHPNCQQQLLTIWYENLSGLREQTVAIKCLVVLVVALGLPFLAIGYWIAPCSRLGKILRSPFMKFVAHAASFIIFLGLLVFNASDRFEGIATLPNITVIDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQIISEGLYAIAVVLSFSRIAYILPANESFGPLQISLGRTVKDIFKFMVLFIMVFLAFMIGMFILYSYYLGAKVNSAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRIINFSKCRRRRLQKDLEMGMGNSKSRQIMKRLIKRYVLKAQVDKENDEVNEGELKEIKQDISSLRYELLEDKSQATEELAVLIHKLSEKLNPSVLRCE

SEQ ID NO: 11 - DNA, 2427 bp, Cavia porcellus; identical to SEQ ID NO: 9

SEQ ID NO: 12 - protein, 836 aa, Cavia porcellus
MREKGRRQAVRGPAFMFNDRGTSLTPEEERFLDAAEYGNIPVVRKMLEESRTLNVNCVDYMGQNALQLAVGNEHLEVTELLLKKENLARIGDALLLAISKGYVRIVEAILNHPGFAASRRLTLSPCEQELQDDDFYAYDEDGTRFSPDITPIILAAHCQKYEVVHMLLLKGARIERPHDYFCKCADCAEKQRHDSFSHSRSRINAYKGLASPAYLSLSSEDPVLTALELSNELAKLANIEKEFKNDYRKLSMQCKDFVVGVLDLCRDSEEVEAILNGDLESTEPLEVHRHKASLSRVKLAIKYEVKKFVAHPNCQQQLLTIWYENLSGLREQTVAIKCLVVLVVALGLPFLAIGYWIAPCSRLGKILRSPFMKFVAHAASFIIFLGLLVFNASDRFEGIATLPNITVIDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQIISEGLYAIAVVLSFSRIAYILPANESFGPLQISLGRTVKDIFKFMVLFIMVFLAFMIGMFILYSYYLGAKVNSAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRIINFSKCRRRRLQKDLEMGMGNSKSRLNLFTQSNSRVFESHSFNSILNQPTRYQQIMKRLIKRYVLKAQVDKENDEVNEGELKEIKQDISSLRYELLEDKSQATEELAVLIHKLSEKLNPSVLRCE

SEQ ID NO: 13 - DNA, 28 bp, Artificial Sequence
  Mouse forward primer
ctaacttttccaaatgcaggaggagaag

SEQ ID NO: 14 - DNA, 28 bp, Artificial Sequence
  Mouse reverse primer
tcgcatgataaaggtagggaacactaga

SEQ ID NO: 15 - DNA, 24 bp, Artificial Sequence
  Rat forward primer
cagtgatgtagagtggaagtttgc

SEQ ID NO: 16 - DNA, 21 bp, Artificial Sequence
  Rat reverse primer
ctccctcattcacacctcagc

SEQ ID NO: 17 - DNA, 29 bp, Artificial Sequence
  Guinea pig forward primer
ggatcattaacttttccaaatgtagaagg

SEQ ID NO: 18 - DNA, 26 bp, Artificial Sequence
  Guinea pig reverse primer
tctcagcacgctgggattcagtttct

SEQ ID NO: 19 - DNA, 27 bp, Artificial Sequence
  TRPC forward primer
acagaattcctgcggggatgcgtgaca

SEQ ID NO: 20 - DNA, 28 bp, Artificial Sequence
  TRPC reverse primer
agcggatcccctcactcacatctcagca

SEQ ID NO: 21 - DNA, 2382 bp, Homo sapiens
atggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctttggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagatattatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatgccaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaagacttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatga

SEQ ID NO: 22 - protein, 793 aa, Homo sapiens [sequence omitted]

SEQ ID NO: 23 - protein, 765 aa, Homo sapiens
MEGSPSLRRMTVMREKGRRQAVRGPAFMFNDRGTSLTAEEERFLDAAEYGNIPVVRKMLEESKTLNVNCVDYMGQNALQLAVGNEHLEVTELLLKKENLARIGDALLLAISKGYVRIVEAILNHPGFAASKRLTLSPCEQELQDDDFYAYDEDGTRFSPDITPIILAAHCQKYEVVHMLLMKGARIERPHDYFCKCGDCMEKQRHDSFSHSRSRINAYKGLASPAYLSLSSEDPVLTALELSNELAKLANIEKEFKNDYRKLSMQCKDFVVGVLDLCRDSEEVEAILNGDLESAEPLEVHRHKASLSRVKLAIKYEVKKLGKILRSPFMKFVAHAASFIIFLGLLVFNASDRFEGITTLPNITVTDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQILSEGLYAIAVVLSFSRIAYILPANESFGPLQISLGRTVKDIFKFMVLFIMVFFAFMIGMFILYSYYLGAKVNAAFTTVEESFKTLFWSIFGLSEVTSVVLKYDHKFIENIGYVLYGIYNVTMVVVLLNMLIAMINSSCQEIEDDSDVEWKFARSKLWLSYFDDGKTLPPPFSLVPSPKSFVYFIMRIVNFPKCRRRRLQKDIEMGMGNSKSRQIMKRLIKRYVLKAQVDKENDEVNEGELKEIKQDISSLRYELLEDKSQATEELAILIHKLSEKLNPSMLRCE

SEQ ID NO: 24 - DNA, 2547 bp, Homo sapiens
atggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagtttgtggctcatcccaactgccagcagcagctcttgacgatctggtatgagaacctctcaggcctaagggagcagaccatagctatcaagtgtctcgttgtgctggtcgtggccctgggccttccattcctggccattggctactggatcgcaccttgcagcaggctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctctggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagattatatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatatcaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaaggcttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatga

SEQ ID NO: 25 - protein, 848 aa, Homo sapiens
MEGSPSLRRMTVMREKGRRQAVRGPAFMFNDRGTSLTAEEERFLDAAEYGNIPVVRKMLEESKTLNVNCVDYMGQNALQLAVGNEHLEVTELLLKKENLARIGDALLLAISKGYVRIVEAILNHPGFAASKRLTLSPCEQELQDDDFYAYDEDGTRFSPDITPIILAAHCQKYEVVHMLLMKGARIERPHDYFCKCGDCMEKQRHDSFSHSRSRINAYKGLASPAYLSLSSEDPVLTALELSNELAKLANIEKEFKNDYRKLSMQCKDFVVGVLDLCRDSEEVEAILNGDLESAEPLEVHRHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVTDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQIISEGLYAIAVVLSFSRIAYILPANESFGPLQISLGRTVKDIFKFMVLFIMVFFAFMIGMFILYSYYLGAKVNAAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRIVNFPKCRRRRLQKDIEMGMGNSKSRLNLFTQSNSRVFESHSFNSILNQPTRYQQIMKRLIKRYVLKAQVDKENDEVNEGELKEIKQDISSLRYELLEDKSQATEELAILIHKLSEKLNPSMLRCE

SEQ ID NO: 26 - protein, 820 aa, Homo sapiens
MEGSPSLRRMTVMREKGRRQAVRGPAFMFNDRGTSLTAEEERFLDAAEYGNIPVVRKMLEESKTLNVNCVDYMGQNALQLAVGNEHLEVTELLLKKENLARIGDALLLAISKGYVRIVEAILNHPGFAASKRLTLSPCEQELQDDDFYAYDEDGTRFSPDITPIILAAHCQKYEVVHMLLMKGARIERPHDYFCKCGDCMEKQRHDSFSHSRSRINAYKGLASPAYLSLSSEDPVLTALELSNELAKLANIEKEFKNDYRKLSMQCKDFVVGVLDLCRDSEEVEAILNGDLESAEPLEVHRHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVTDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQIISEGLYAIAVVLSFSRIAYILPANESFGPLQISLGRTVKDIFKFMVLFIMVFFAFMIGMFILYSYYLGAKVNAAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRIVNFPKCRRRRLQKDIEMGMGNSKSRQIMKRLIKRYVLKAQVDKENDEVNEGELKEIKQDISSLRYELLEDKSQATEELAILIHKLSEKLNPSMLRCE

(19) United States
(12) Patent Application Publication
(10) Pub. No.: US 2017/0202932 A1
(43) Pub. Date: Jul. 20, 2017

(54) METHODS FOR INHIBITING NEURON APOPTOSIS AND NECROSIS

(71) Applicant: NEWSOUTH INNOVATIONS PTY LIMITED, Sydney (AU)

(72) Inventors: Gary David HOUSLEY, Connells Point (AU); Youngsoo Kim, Randwick (AU); Paul Page BERTRAND, Rosanna (AU); Andrew MOORHOUSE, Kensington (AU); Ann Chi Yan Wong, Rhodes (AU); Amanda Jayne CRAIG, Randwick (AU); John POWER, Randwick (AU); Matthias KLUGMANN, Randwick (AU); Arun KRISHNAN, Randwick (AU); Renee MORRIS, Bondi (AU)

(21) Appl. No.: 15/467,404

(22) Filed: Mar. 23, 2017

Related U.S. Application Data
(63) Continuation of application No. 14/412,793, filed on Jan. 5, 2015, now abandoned.

(30) Foreign Application Priority Data
Jul. 6, 2012 (AU) 2012902920

(51) Int. Cl. A61K 38/48; A61K 31/353; A61K 45/06

(52) U.S. Cl. CPC A61K 38/482; C12Y 304/21068; A61K 45/06; A61K 31/353

(57) ABSTRACT

The present invention relates generally to methods for inhibiting neuron apoptosis and necrosis associated with excess glutamate release.

### CROSS-REFERENCE TO RELATED APPLICATIONS

This application is a Continuation application of U.S. application Ser. No. 14/412,793, filed Jan. 4, 2015, which claims priority to International Application No. PCT/AU2013/0900732, filed Jul. 5, 2013, which claims priority to Australian Patent Application No. 2012902920, filed Jul. 6, 2012. The disclosures of the priority applications are incorporated in their entirety herein by reference.

### BACKGROUND

Field of the Invention

The present invention relates generally to methods for inhibiting neuron apoptosis and necrosis associated with excess glutamate release.

Stroke refers to the loss of blood supply to the brain, resulting either from infarct or hemorrhage. Stroke is one of the leading causes of death and disability in many countries. Only 50% of hemorrhagic stroke sufferers and 85% of ischaemic stroke victims survive. With complete recovery at only around 10%, the majority of stroke patients sustain long-term debilitating impairments to their physical, mental and social wellbeing.

Description of Related Art

The primary treatment for ischaemic stroke is to target the blockage of blood supply with fibrinolytic therapy in a critical (“golden”) window of a few hours following onset of symptoms, with antithrombotic therapy for secondary prevention. However, the pathophysiology of brain infarct/stroke involves apoptotic and necrotic cell death pathways that are induced at the immediate onset of stroke and subsequently. For example, the sequelae of the brain tissue response to ischaemic injury invariably includes initial glutamatergic excitotoxicity arising from release of excess glutamate from neurons and glia. This causes widespread activation of synaptic and extra-synaptic glutamate receptors, in particular the NMDA subtype, which have a high Ca²⁺ permeability. Excessive and sustained elevation of cytosolic Ca²⁺ instigates a series of downstream biochemical pathways that trigger apoptosis and cell death in the neurons and glia. The neuroinflammatory response, reflected by the microglial invasion into the region of the infarct is associated with release of pro-apoptotic factors such as a range of cytokines (IL-1β and TNF-α). Restoration of perfusion to the brain only partially attenuates on-going tissue damage. Thus, the lesion radiates out over days and weeks in the penumbral region of the infarct.

Experimental strategies for treating stroke injury have typically targeted principal upstream elements, particularly the NMDA receptors (considered to be the main receptor involved in triggering significant Ca²⁺ entry into cells), as well as downstream processes within the apoptosis cascade, such as CREB elements, caspases and members of the Bcl-2 family (Bax), HIF1a, p53 and a plethora of associated pro-apoptotic signalling partners. Many potential treatments of ischaemic stroke have targeted Ca²⁺ entry, either with antagonists of the NMDA glutamate receptor (e.g. MK-801/dizocilpine and CGS19755) and its allosteric binding sites (e.g. gavestinel, targeting the glycine binding site, and Mg²⁺ for Mg²⁺ block), or voltage-gated Ca²⁺ channels. Other trials have investigated the neuroprotective efficacy of AMPA receptor antagonists, GABA receptor agonism, and free radical scavengers. All have been unsuccessful at providing clinical efficacy, typically failing at clinical trial due to adverse neuropsychological events. Thus, there is a need for improved methods of treating stroke injury and other similar brain injury.

### SUMMARY

The present invention relates to methods for inhibiting apoptosis or necrosis of neurons in a subject, comprising administering a TRPC3 inhibitor to the subject. In some embodiments, the subject is experiencing or has experienced an event that results in the release of excess glutamate in the brain. In particular embodiments, the event is a stroke, an epileptic seizure, a head trauma, severe blood loss, cardiac arrest, or other ischaemic event. For example, in one embodiment of the present invention, the event that results in the release of excess glutamate in the brain is a stroke, such as an ischaemic stroke. In a particular example, the stroke is a hindbrain stroke. In some embodiments of the method, the neurons are in the cerebellum or midbrain of the subject. In a particular example, the neurons are Purkinje cells.

The present invention also relates to methods for treating or preventing brain injury associated with stroke, an epileptic seizure, a head trauma, severe blood loss, cardiac arrest, or other ischaemic event in a subject, comprising administering a TRPC3 inhibitor to the subject. For example, provided are methods for treating or preventing brain injury associated with ischaemic stroke. Also provided are methods for treating or preventing brain injury associated with a hindbrain stroke.

In some embodiments of the methods of the present invention, an additional therapeutic agent is also administered to the subject. For example, another neuroprotective agent, a thrombolytic agent, insulin, an antiplatelet agent, anticoagulants and/or a procoagulant can be administered to the subject. In a particular embodiment, tissue plasminogen activator is administered to the subject. In such methods, the TRPC3 inhibitor can be administered to the subject before, at the same time, or after the additional therapeutic agent is administered to the subject.

The present invention is also directed to methods for preventing or inhibiting apoptosis or necrosis of neurons in a subject that is experiencing or has experienced a stroke, comprising administering to the subject a thrombolytic agent and a TRPC3 inhibitor. Also provided are methods for treating a stroke in a subject, comprising administering to the subject a thrombolytic agent and a TRPC3 inhibitor; and methods for preventing or treating brain injury associated with a stroke in a subject, comprising administering to the subject a TRPC3 inhibitor and a thrombolytic agent. In some embodiments of these methods, the TRPC3 inhibitor and the thrombolytic agent are administered to the subject at the same time. In other examples, the TRPC3 inhibitor is administered to the subject after the thrombolytic agent is administered to the subject. In one embodiment, the stroke is an ischaemic stroke. In a particular embodiment, the stroke is a hindbrain stroke.

In the methods of the present invention, the TRPC3 inhibitor can be administered to the subject by any route. In some instances, the route is selected from among a parenteral, intravenous, intraarterial, intramuscular, intracranial, intraorbital, nasal, or intraventricular route.

In particular embodiments of the methods of the present invention, the TRPC3 inhibitor selectively inhibits the formation, activation or activity of TRPC3c channels. In further embodiments, the TRPC3 inhibitor selectively inhibits the formation, activation or activity of TRPC3 channels. In another embodiment, the TRPC3 inhibitor inhibits the formation, activation or activity of TRPC3 channels and one or more other TRPC channels. In some instances, the TRPC3 inhibitor is a small molecule, protein or nucleic acid molecule. For example, the TRPC3 inhibitor can be a tyrosine kinase inhibitor. In particular embodiments, the TRPC3 inhibitor is selected from the group consisting of genistein (4′, 5, 7-trihydroxyisoflavone or 5, 7-dihydroxy-3-(4-hydroxyphenyl)-4H-1-benzopyran-4-one), PP2 (3-(4-chlorophenyl) 1-(1,1-dimethylethyl)-1H-pyrazolo[3,4-d]pyrimidin-4-amine), 2-aminoethoxydiphenylborane (2-APB), SKF96365, bis(trifluoromethyl)pyrazoles (BTPs), such as 4-methyl-4′-[3,5-bis(trifluoromethyl)-1H-pyrazol-1-yl]-1,2,3-thiadiazole-5-carboxanilide (BTP2), ethyl-1-(4-(2,3,3-trichloroacrylamide)phenyl)-5-(trifluoromethyl)-1H-pyrazole-4-carboxylate (Pyr3) and related compounds, norgestimate, erbstatin-analog, herbimycin and lavendustin A. For example, in some embodiments of the present invention, the TRPC3 inhibitor is Pyr3. In other embodiments, the TRPC3 inhibitor is genistein.

In the methods of the present invention, the subject to which the TRPC3 inhibitor is administered can be a human or non-human subject. In some examples, the subject is a human subject. In other examples, the subject is a non-human subject, such as a non-human primate, monkey, mouse, cow, sheep, dog, cats, horse, bird or pig.

The present invention is also directed to compositions comprising a TRPC3 inhibitor for use in inhibiting apoptosis or necrosis of neurons; compositions comprising a TRPC3 inhibitor for use in treating or preventing brain injury associated with stroke, an epileptic seizure, a head trauma or severe blood loss; and compositions comprising a TRPC3 inhibitor for use in treating stroke. In some aspects, the composition further comprises an additional therapeutic agent. For example, the compositions of the present invention can include a neuroprotective agent, a thrombolytic agent, insulin, an antiplatelet agent, an anticoagulants and/or a procoagulant. In particular embodiments, the compositions include a thrombolytic agent, such as tissue plasminogen activator.

The present invention is also directed to uses of a TRPC3 inhibitor for the preparation of a medicament for inhibiting apoptosis or necrosis of neurons; uses of a TRPC3 inhibitor for the preparation of a medicament for the treatment or prevention of brain injury associated with stroke, an epileptic seizure, a head trauma, severe blood loss, cardiac arrest, or other ischaemic events; and uses of a TRPC3 inhibitor for the preparation of a medicament for the treatment of stroke. In particular embodiments, the medicament further comprises an additional therapeutic agent. For example, the medicament can include a neuroprotective agent, a thrombolytic agent, insulin, an antiplatelet agent, an anticoagulants and/or a procoagulant. In particular embodiments, the medicament includes a thrombolytic agent, such as tissue plasminogen activator.

### BRIEF DESCRIPTION OF THE DRAWINGS

Embodiments of the present invention are further described herein, by way of non-limiting example only, with reference to the accompanying drawings.

FIG. 1 is a schematic of regions of the mouse TRPC3b and TRPC3c polypeptides encoded by exons 8 to 10, showing the predicted calmodulin/IP₃ receptor binding (CIRB) domain.

FIG. 2 represents the results of experiments showing TRPC3 isoform expression in the brain. A: Agarose gel electrophoresis showing TRPC3b (upper) and TRPC3c (lower) RT-PCR amplicons from different brain regions of mouse, rat and guinea pig. B: Semi-quantification of the expression of TRPC3c cDNA amplicon fluorescence intensity on the agarose gel, as a proportion of the combined TRPC3c and TRPC3b signals, as shown in A. Regional differences in TRPC3c expression are apparent (* indicates p<0.05, Dunn's pairwise post-hoc comparison of ranked data from ANOVA) with highest relative levels in cerebellum, followed by midbrain. C: Immunolabelling of the TRPC3 protein in the mouse cerebellum, showing the high-level of staining in the Purkinje neurons including their neurite projections into the molecular layer (PCL, Purkinje cell layer; IGL, internal granule cell layer; ML, molecular layer; m, mouse; r, rat; gp, guinea-pig).

FIG. 3 represents the results of Western blot and immunohistochemistry of HEK293 cells expressing recombinant TRPC3b and TRPC3c. A: Whole-cell lysate samples of transfected and untransfected HEK293 cells separated by 10% SDS-PAGE gel, blotted onto PVDF membrane, and probed for TRPC3 protein with rabbit anti-TRPC3 antibody show as ˜75 kDa protein species. The TRPC3c isoform has a slightly smaller size, which is predicted based on the loss of 28 aa, encoded by exon 9 (equivalent to ˜3.1 kDa). B: Detection of actin in the same blot after anti-TRPC3 strip-off provides a control for protein loading (43 kDa). C: TRPC3 immunodetection of the membrane-bound fraction labeled with NHS-biotin and purified by adsorption onto NeutrAvidin beads, separated by a 10% SDS-PAGE gel followed by Western blotting with anti-TRPC3 antibody, ˜75 kDa. D: TRPC3b and TRPC3c expression in transfected HEK293 cells detected with anti-TRPC3 antibody by immunofluorescence confocal microscopy. The images are consistent with lower expression of TRPC3c as indicated the Western blot above (A). TRPC3 specific immunolabelling was localised to the plasma membrane and cytoplasm in the transfected cells; untransfected cells (control) were unlabelled.

FIG. 4 represents the results of whole-cell voltage clamp recordings of HEK293 cells expressing recombinant TRPC3b or TRPC3c channels. A: Example of the larger currents produced by the TRPC3c transfected cells (currents activated by bath application of carbachol (CCh; 100 μM). The currents were blocked by pre-incubation with genistein (10 mins; 200 μM). Example shows block of TRPC3c current; Vh=−50 mV; dashed lines indicate zero-current. B: Current/voltage relationships (I/Vs; mean±s.e.m.) for TRPC3b and TRPC3c (1=control ramp prior to CCh; 2=ramp during CCh response; 2-1 represents the isolated I_{TRPC3} I/V (trace 2-trace 1). The reversal potential (Erev) of I_{TRPC3} was close to 0 mV for both isoforms, indicating that the ion selectivity of the two channel isoforms was similarly non-selective. C: Mean peak whole-cell current responses for both isoforms of TRPC3 channels, genistein block for each, and control data (untransfected cells). ***P<0.001; two-way ranked ANOVA, Holm-Sidak multiple pairwise comparisons).

FIG. 5 represents the results of a single channel patch-clamp recording of HEK293 cells expressing recombinant TRPC3 channels. A: Current traces of HEK293 cells expressing TRPC3b and TRPC3c channels. Each cell group is from the same patch recording and contains four experimental modes as shown (i, ii, iii and iv). B: Representative single channel current transients in cell attached mode shown at high temporal resolution, with CCh (100 μM), as for Aii; 0=closed state, 1=open state. C: Mean channel opening frequency of membrane patches containing TRPC3b and TRPC3c channels, as well as control patches (no recombinant TRPC3 channel). “n.s.” indicates that the differences were not significant (p>0.05).

FIG. 6 represents the results of ratiometric Ca²⁺ imaging of a field of HEK293 cells expressing recombinant TRPC3 channels using Indo-1 Ca²⁺ indicator dye. Cells were superfused with nominal Ca²⁺-free solution followed by application of carbachol (100 μM) which causes release of stored Ca²⁺ via IP₃R activation. Once released Ca²⁺ has been eliminated from the cell, the extracellular Ca²⁺ is returned to the bath, enabling TRPC3 channel-mediated Ca²⁺ entry (arrows). A: Greater Ca²⁺ entry in TRPC3c expressing cells compared with TRPC3b expressing cells or genistein block (200 μM; throughout the experiment). B: Mean peak [Ca²⁺]ᵢ arising from TRPC3b- and TRPC3c-mediated Ca²⁺ entry, genistein block and control (untransfected cell) data. ***, p<0.001; two-way ranked ANOVA, Holm-Sidak multiple pairwise comparison, and Mann-Whitney rank sum test.

FIG. 7 represents the results of fluo-4AM Ca²⁺ imaging of HEK293 cells co-expressing recombinant TRPC3 channels and mGluR1. A: Rise in [Ca²⁺]ᵢ from Ca²⁺ store release is shown for a single cell with application of the mGluR1 agonist DHPG (200 μM) in Ca²⁺ free solution. Fluorescence signal declines as the Ca²⁺ is extruded from the cell, and then rises again with TRPC3-mediated Ca²⁺ entry upon return of Ca²⁺ to the bath (arrows). Greater Ca²⁺ entry in TRPC3c expressing cells compared with TRPC3b expressing cells, or genistein block (200 μM; throughout the experiment). F₀ represents the Ca²⁺ signal just prior to DHPG application. B: Relative mean peak [Ca²⁺]ᵢ (F/Fo) arising from TRPC3b and TRPC3c-mediated Ca²⁺ entry, genistein block, and control (expression of mGluR1 only, no TRPC3). ***, p<0.001; one-way ranked ANOVA, Holm-Sidak multiple pairwise comparison.

FIG. 8 represents the results of whole-cell voltage-clamp recordings of DHPG-evoked inward currents in Purkinje cells. The mGluR agonist DHPG (50 μM) was applied onto the Purkinje cell's dendrites by pressure application (50 ms, 70 kPa) through a patch pipette. A: Bright field image of the cerebellar slice shows the recording pipette (r) on the Purkinje cell (PC) soma and drug pipette (d) containing DHPG (50 μM) positioned over the dendritic field (see B). B: Fluorescence image of the Purkinje cell loaded with Alexa 594 via the patch-clamp pipette (r). C: The current (Vm −70 mV) evoked by DHPG before and after bath application of the TRPC3 channel blocker genistein (100 μM). Arrowhead indicates the timing of DHPG application. D: Time course plot showing the normalized integrated area of repeated (3 minute intervals) DHPG-evoked currents before and during application of genistein (indicated by bar). Mean±SEM (n=3) responses.

FIG. 9 represents the result of comparing the effect of ischaemia (oxygen glucose deprivation—OGD) on mouse cerebellar brain tissue in the absence (control) or presence of the TRPC3 channel blocker genistein (200 μM). In the control brain slices, OGD produced oedema, particularly in the purkinje cell layer (PCL). This was more extensive after 30 minutes OGD, compared with 15 minutes OGD. O minutes OGD showed minimal tissue disruption (with or without genistein). Genistein provided protection from the neuronal loss and oedema in the PCL—both at 15 mins and 30 mins. There was also reduced propidium iodide fluorescence in the granule cell layer (GCL) and in the molecular layer (ML). Confocal laser scanning microscopy with 561 nm excitation.

### DETAILED DESCRIPTION OF A PREFERRED EMBODIMENT

The articles “a” and “an” are used herein to refer to one or to more than one (i.e., to at least one) of the grammatical object of the article. By way of example, “an antimicrobial agent” means one antimicrobial agent or more than one antimicrobial agent.

In the context of this specification, the term “about” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “TRPC channel” refers to a canonical transient receptor potential channel TRPC channels are multimeric Ca²⁺ permeable non-selective cation channels, and reference to a TRPC channel includes reference to both homomeric and heteromeric channels formed by one or more of the TRPC1, TRPC2, TRPC3, TRPC4, TRPC5, TRPC6 and TRPC7 polypeptides, including isoforms and splice variants thereof. Reference to a TRPC channel includes reference to human TRPC channels as well as non-human TRPC channels, including, but not limited to, mouse, rat, guinea pig, dog, horse, cat, sheep, monkey and chimpanzee TRPC channels.

As used herein, the term “TRPC3 channel” refers to a Ca²⁺ permeable non-selective cation channel formed by a TRPC3 polypeptide. A TRPC3 channel can be homomeric (i.e. formed only by TRPC3 polypeptides) or heteromeric (i.e. formed by at least one TRPC3 polypeptide and one or more different TRPC polypeptides, such as a TRPC1, TRPC2, TRPC4, TRPC5, TRPC6 or TRPC7 polypeptide). TRPC3 channels include human TRPC3 channels as well as non-human TRPC3 channels, such as mouse, rat, guinea pig, dog, horse, cat, sheep, monkey and chimpanzee TRPC3 channels, and can be formed by any TRPC3 polypeptide. TRPC3 polypeptides include polypeptides encoded by the full length transcript from a TRPC3 gene (e.g. TRPC3b) as well polypeptides encoded by alternatively spliced transcripts (e.g. TRPC3c or TRPC3a). Exemplary TRPC3 polypeptides include, but are not limited to, human TRPC3a (Genbank Acc. No. NP_001124170); human TRPC3b (SEQ ID NO:22; Genbank Acc. No. BAF76423, and SEQ ID NO:25; Genbank Acc. No. AAH93684); human TRPC3c human (SEQ ID NOS: 23 and 26; as predicted from homology to mouse, rat and guinea-pig TRPC3 exon 9 splice sites); mouse TRPC3b (SEQ ID NO:4; Genbank Acc. No. BAC37961); mouse TRPC3c (SEQ ID NO:2; Genbank Acc. No. ACO07350); rat TRPC3b (SEQ ID NO:8; Genbank Acc. No. NP068539); rat TRPC3c (SEQ ID NO:6; Genbank Acc. No. AEK22122); guinea pig TRPC3b (SEQ ID NO:12; Genbank Acc. No. NP001166502) and guinea pig TRPC3c (SEQ ID NO:10; Genbank Acc. No. ACO07348) polypeptides.

As used herein, the term “TRPC3b channel” refers to a Ca²⁺ permeable non-selective cation channel formed with a TRPC3b polypeptide. A TRPC3b channel can be homomeric (i.e. formed only by TRPC3b polypeptides) or may be heteromeric (i.e. formed by at least one TRPC3b polypeptide and one or more different TRPC polypeptides, such as a TRPC3c, TRPC1, TRPC2, TRPC4, TRPC5, TRPC6 or TRPC7 polypeptide). TRPC3b channels include human TRPC3b channels as well as non-human TRPC3b channels, such as mouse, rat, guinea pig, dog, horse, cat, sheep, monkey and chimpanzee TRPC3b channels, as well as any allelic variants, including splice variants.

As used herein, a “TRPC3b polypeptide” or “TRPC3b” is a polypeptide having a sequence of amino acids that is the same as the sequence of amino acids encoded by a full length, non-spliced transcript of a TRPC3 gene, such as the human TRPC3 gene (Genbank Acc. No. NG030368). Accordingly, TRPC3b polypeptides have a sequence of amino acids encoded by exons 1, 2, 3, 4, 5, 6, 7, 8, 9, 10, 11 and 12. Exemplary TRPC3b polypeptides include, but are not limited to, human (SEQ ID NO:25), mouse (SEQ ID NO:4), rat (SEQ ID NO:8) and guinea pig (SEQ ID NO:12) TRPC3b polypeptides.

As used herein, the term “TRPC3c channel” refers to a Ca²⁺ permeable non-selective cation channel formed by a TRPC3c polypeptide. A TRPC3c channel can be homomeric (i.e. be formed only by TRPC3c polypeptides) or may be heteromeric (i.e. formed by at least one TRPC3c polypeptide and one or more different TRPC polypeptides, such as a TRPC3b, TRPC1, TRPC2, TRPC4, TRPC5, TRPC6 or TRPC7 polypeptide). TRPC3c channels include human TRPC3c channels as well as non-human TRPC3c channels, such as mouse, rat, guinea pig, dog, horse, cat, sheep, monkey and chimpanzee TRPC3c, as well as any allelic variants, including splice variants.

As used herein, a “TRPC3c polypeptide” or “TRPC3c” is a polypeptide that lacks the amino acids corresponding to the amino acids encoded by exon 9 of a TRPC3 gene, such as the human TRPC3 gene (Genbank Acc. No. NG030368). When expressed from a TRPC3 gene in its natural environment (i.e. from an endogenous TRPC3 gene), a TRPC3c polypeptide is the result of alternative splicing to remove exon 9. However those skilled in the art will understand that TRPC3c polypeptides can be recombinantly expressed without alternative splicing by, for example, introducing a nucleic acid molecule having a sequence corresponding to the cDNA of the alternatively spliced transcript into a cell. Exemplary TRPC3c polypeptides include, but are not limited to, human (SEQ ID NOS: 23 and 36; as predicted from homology to mouse, rat and guinea-pig TRPC3 exon 9 splice sites), mouse (SEQ ID NO:2; GenBank: Accession: ACO07350.1), rat (SEQ ID NO:6; GenBank: Accession: AEK22122.1) and guinea pig (SEQ ID NO:10; GenBank: Accession: ACO07348.1) TRPC3c polypeptides.

As used herein, a “TRPC3 inhibitor” or an “inhibitor of TRPC3” or grammatical variations thereof refers to an agent that inhibits the expression or activity of a TRPC3 polypeptide or channel, including variants or isoforms thereof, such as TRPC3c and TRPC3b. A TRPC3 inhibitor can selectively inhibit a TRPC3 polypeptide and/or TRPC3 channel, or can inhibit a TRPC3 polypeptide and/or TRPC3 channel and also inhibit one or more other polypeptides and/or channels, such as one or more other TRPC polypeptides and/or channels. The inhibition may be to an extent (in magnitude and/or spatially), and/or for a time, sufficient to produce the desired effect. Inhibition may be prevention, retardation, reduction or otherwise hindrance of TRPC3 expression and/or activity. Such inhibition may be in magnitude and/or be temporal or spatial in nature. Inhibition of expression of TRPC3 can be assessed using methods well known in the art to measure transcription and/or protein production. Inhibition of the activity of TRPC3 can be assessed by, for example, determining the ability of a TRPC3 polypeptide to form a channel and/or the ability of a TRPC3 channel to facilitate cation flux. Methods to assess TRPC3 activity by assessing TRPC3 channel conductance are described herein and can be used to determine the level of inhibition of TRPC3 activity resulting from a TRPC3 inhibitor. The expression and/or activity of TRPC3 can be inhibited by an agent by at least or about 10%, 15%, 20%, 25%, 30%, 35%, 40%, 45%, 50%, 55%, 60%, 65%, 70%, 75%, 80%, 85%, 90%, 95% or more compared to the expression and/or activity of TRPC3 in the absence of the agent. A TRPC3 inhibitor may be specific or selective for TRPC3 or may be capable of inhibiting the expression or activity of one or more TRPC polypeptides or channels in addition to TRPC3. Furthermore, a TRPC3 inhibitor may act directly or indirectly on TRPC3. Accordingly the inhibitor may operate directly or indirectly on a TRPC3c polypeptide or channel, a TRPC3 mRNA or gene, or alternatively act via the direct or indirect inhibition of any one or more components of a TRPC3-associated pathway. Such components may be molecules activated, inhibited or otherwise modulated prior to, in conjunction with, or as a consequence of TRPC3 polypeptide or channel activity.

As used herein, a “TRPC3c activity” or an “activity of TRPC3c” refers to any activity associated with a TRPC3c polypeptide and/or TRPC3c channel, including, but not limited to, the ability of a TRPC3c polypeptide to form a channel, the ability of a TRPC3c channel to be activated and the ability of a TRPC3c channel to facilitate cation flux. Similarly, a “TRPC3 activity” or an “activity of TRPC3” refers to any activity associated with a TRPC3 polypeptide and/or TRPC3 channel, including, but not limited to, the ability of a TRPC3 polypeptide (including TRPC3c and TRPC3b polypeptides) to form a channel, the ability of a TRPC3 channel (including TRPC3c and TRPC3b channels) to be activated and the ability of a TRPC3c channel (including TRPC3c and TRPC3b channels) to facilitate cation flux.

The term “inhibiting” and variations thereof such as “inhibition” and “inhibits” as used herein in relation to apoptosis or necrosis of neurons, or the formation, activity or activation or formation of TRPC channels (e.g. TRPC, TRPC3 or TRPC3c channels), means complete or partial inhibition of apoptosis or necrosis of neurons or complete or partial inhibition of the formation, activity or activation of TRPC channels. The inhibition may be to an extent (in magnitude and/or spatially), and/or for a time, sufficient to produce the desired effect. Inhibition may be prevention, retardation, reduction or otherwise hindrance of apoptosis or necrosis of neurons or of the formation, activity or activation of TRPC channels. Such inhibition may be in magnitude and/or be temporal or spatial in nature. Inhibition of the apoptosis or necrosis of neurons by an agent (i.e. a TRPC3 inhibitor) can be assessed by measuring necrosis or apoptosis in the presence and absence of the agent following an event that would normally trigger apoptosis or necrosis, such as, for example, oxygen deprivation. The apoptosis or necrosis of neurons can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to the apoptosis or necrosis of neurons that have not been exposed to the agent. Inhibition of the activation, activity or formation of TRPC channels by an agent (i.e. a TRPC3 inhibitor) can be assessed by measuring, for example, cation flux, membrane conductance, and/or Ca²⁺ entry into cells in the presence and absence of the agent. The activation, activity or formation of TRPC channels can be inhibited by the agent by at least or about 10%, 15%, 20%, 25%, 30%, 35%, 40%, 45%, 50%, 55%, 60%, 65%, 70%, 75%, 80%, 85%, 90%, 95% or more compared to the activation or formation of TRPC channels that have not been exposed to the agent.

As used herein, the term “selectively inhibits” with reference to a TRPC3 inhibitor means that the inhibitor inhibits the formation, activation or activity of a recited TRPC channel but does not inhibit the formation, activation or activity of one or more non-recited channels. For example, a TRPC3 inhibitor that selectively inhibits TRPC3 channels inhibits the formation, activation or activity of a TRPC3 channel (including a TRPC3c channel and/or a TRPC3b channel) but does not inhibit the formation, activation or activity of a TRPC1, TRPC2, TRPC4, TRPC5, TRPC6, or TRPC7 channel. In another example, a TRPC3 inhibitor that selectively inhibits TRPC3c channels inhibits the formation, activation or activity of a TRPC3 channel but does not inhibit the formation, activation or activity of a TRPC3b, TRPC1, TRPC2, TRPC4, TRPC5, TRPC6, or TRPC7 channel.

As used herein, the phrase “excess release of glutamate” refers to the release of an amount of glutamate in the brain that is greater than the amount of glutamate released under normal conditions, i.e. an amount of glutamate released in the brain that is greater than the amount of glutamate normally released in the brain of a subject prior to that subject experiencing an event associated with excess glutamate release, such as a stroke, epileptic seizure, head trauma, severe blood loss, cardiac arrest or other ischaemic incident. An excess release of glutamate in the brain is characterized by a release of more glutamate than is released in a brain under normal conditions, and which is sufficient to typically induce pathological changes in brain tissues.

As used herein the term “expression” may refer to expression of a polypeptide or protein, or to expression of a polynucleotide or gene, depending on the context. Expression of a polynucleotide may be determined, for example, by measuring the production of RNA transcript levels. Expression of a protein or polypeptide may be determined, for example, by immunoassay using an antibody(ies) that bind with the polypeptide.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As used herein, a “subject” includes human and non-human animals, including, for example, non-human primates, monkeys, mice, cows, sheep, dogs, cats, horses, birds and pigs.

The present invention is related to methods of inhibiting neuron necrosis and apoptosis associated with excess glutamate release and glutamatergic excitotoxicity in the brain. Accordingly, the methods can be used to treat brain injury associated with stroke, epilepsy, head trauma (such as contusions and blunt force trauma), and blood loss, or other ischaemic events, such as cardiac arrest or vascular surgery. The methods of the present invention involve administration of an inhibitor of a canonical Transient Receptor Potential (TRPC) ion channel, in particular a TRPC3c channel.

### TRPC3

Canonical Transient Receptor Potential (TRPC) channels are part of the TRP channel superfamily that form cation channels and that can be activated by range of various mechanisms. The TRPC channel family contains 7 members: TRPC1, TRPC2 (a pseudogene in humans), TRPC3, TRPC4, TRPC5, TRPC6 and TRPC7, which are widely expressed in the brain and have been shown to be involved in various aspects of neuronal development, such as proliferation, differentiation, morphogenesis and synaptogenesis. TRPC1, TRPC4 and TRPC5 form channels following activation primarily by Ca²⁺ store depletion, while TRPC3, TRPC6 and TRPC7 form channels following activation primarily by receptor stimulation, although both mechanisms are often involved in the physiological setting.

Activation of TRPC channels facilitates Ca²⁺ entry across the membrane through the TRPC channel resulting in an increase in intracellular Ca²⁺. Regulation of Ca²⁺ levels through these and other channels is critical for a wide range of functions, including gene regulation, muscle contraction, neurosecretion, neuronal excitability, neuronal proliferation, synaptic plasticity and neuronal apoptosis. TRPC Ca²⁺ signalling instigates a host of cellular responses, which, depending upon the context, can either be integral to the physiology of the cell, or drive detrimental actions at the cell and tissue level.

TRPC3 channels are the putative effector of the cation conductance coupled to metabotropic glutamate receptors (mGluR), P2Y, mAChR and substance P metabotropic receptors. TRPC3 ion channels can be activated by both diacylglycerol (DAG), via phospholipase C (PLC) and by cytosolic allosteric protein-protein regulation. PLC is engaged by a broad range of Gα/q protein-coupled receptors (GPCR-PLCβ), and by receptor tyrosine kinase (Trk-PLCγ) signal transduction. PLC-mediated cleavage of phospholipid phosphatidylinositol 4,5-bisphosphate (PiP₂) into DAG and inositol 1,4,5-trisphosphate (IP₃) enables DAG to diffuse in the plasma membrane to the TRPC3 channel, while IP₃ diffuses through the cytoplasm to separately activate IP₃ receptor-gated Ca²⁺ stores in the endoplasmic reticulum. Thus in neurons, GPCR and receptor tyrosine kinase activation (such as via mGluR, P2Y receptors, mAChR, neurotrophin-mediated Trk signalling) can result in multiplexing of Ca²⁺ signalling via direct Ca²⁺ entry through a common TRPC3 channel effector. In addition, Na⁺ entry depolarizes the cells, enabling parallel Ca²⁺ entry through other pathways, such as NMDA receptors and voltage-gated Ca²⁺ channels.

An intracellular regulatory motif in the C-terminal region of the TRPC3 subunit, designated the Ca²⁺-calmodulin and the IP₃ receptor binding (CIRB) domain, confers negative Ca²⁺ feedback regulation to TRPC3 ion channels. At nominal cytosolic Ca²⁺ levels, the Ca²⁺-calmodulin complex bound to the CIRB domain inhibits spontaneous TRPC channel activity (independent of receptor-mediated activation). Reduction in cytosolic Ca²⁺ reduces the CIRB-calmodulin binding affinity and increases the open probability of the TRPC3 channels. The IP₃ receptor binding site of the CIRB domain (CIRB-IP₃R binding) enables direct protein-protein interaction that regulates TRPC3 channel activation.

TRPC3 has been implicated in neuronal development and protection. For example, a developmental switch in the cerebellum up-regulates TRPC3 compared with the other TRPC isoforms in the rat shortly after birth. In this animal model, expression of TRPC3 and the TrkB receptor for brain derived neurotrophic factor (BDNF) show almost complete overlap during the early post-natal development of the olfactory bulb, cerebral cortex, amygdala, pons and cerebellar Purkinje neurons. In pontine neurons, BDNF-activated non-selective cation currents have been attributed to TrkB receptor-PLCγ1—DAG-mediated activation of TRPC3 ion channels. TRPC3 expression has also been associated with the development of the dendritic arbor of Purkinje neurons and TRPC3 and TRPC6 have been shown to contribute to BDNF-mediated protection of cerebellar granule cells from apoptosis by Ca²⁺⁻ signal-dependent CREB activation. Down-regulating TRPC3 or TRPC6 in neonatal rat cerebellar granule cells induced apoptosis, which could be rescued by overexpressing TRPC3 or TRPC6.

In cerebellar Purkinje neurons where TRPC3 expression is dominant, constitutive activation of TRPC3 channels has been shown to underlie cerebellar ataxias. The moonwalker mouse (Mwk), which provides a principal model of cerebellar ataxia, has a gain-of-function point mutation in the TRPC3 gene that alters channel gating. This mouse model exhibits profound impairment of Purkinje neuron dendrite development and loss of these neurons. Recently, it has been determined that TRPC3 is the sole ion channel effector for metabotropic glutamate receptor (mGluR)-mediated slow mixed-cation excitatory postsynaptic conductance (sEPSC) in the Purkinje neurons, and TRPC3 knockout mice that lack TRPC3 expression are deficient in sEPSC in the Purkinje neurons.

To date, TRPC3 has not been considered as a target for neuroprotection in the context of stroke or other conditions associated with excess glutamate release in the brain. Rather, N-methyl-D-aspartate (NMDA) receptor channels and α-amino-3-hydroxy-5-methyl-4-isoxazolepropionic acid (AMPA) receptor channels, which are activated by glutamate and known to be involved in excitotoxic cell death, have been the targets. This is likely because the Ca²⁺ entry and depolarization resulting from TRPC3 channel activation has until now been considered to be at sufficiently low levels as to be physiologically insignificant in the context of stroke and other glutamate-associated diseases and conditions. Hence, in a recent review of TRPC channel physiology, it is noted that TRPC3 channels have a relatively low selectivity for Ca²⁺ over Na⁺ (P_{Na}/P_{Ca}=1/1.5) (Clapham et al. (2001) Nature Reviews Neuroscience 2. 387-396). In contrast, P_{Na}/P_{Ca} for NMDA receptors is approximately ⅕ (Jahr C. E., Stevens C. F. (1993) PNAS U.S.A. 90, 11573-11577). Thus, given comparable conductances of approximately 50-70 pS (see e.g., Stern et al. (1992) Proc. R. Soc. Lond. B 250. 271-277 and Clapham et al. (2001) Nature Reviews Neuroscience 2. 387-396), activation of NMDA receptors would permit approximately 3 times more Ca²⁺ into neurons.

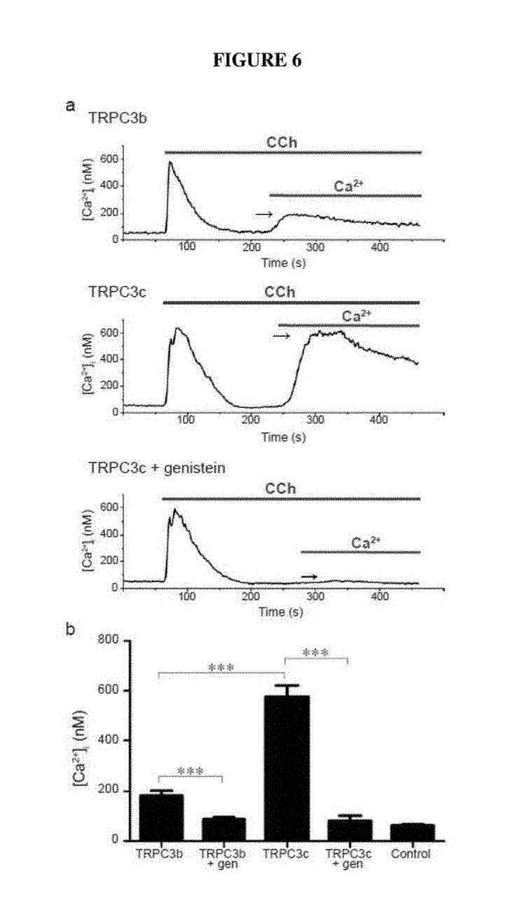

However, as demonstrated herein for the first time, a newly-identified TRPC3c ion channel isoform facilitates approximately 3 times the amount of Ca²⁺ entry into cells compared to that observed with the TRPC3b variant considered by Clapham et al. (see FIG. 6b). This provides the first evidence that glutamate-induced, TRPC3 channel-mediated Ca²⁺ entry is significantly more potent in the brain, in particular the cerebellum and the brainstem, than previously appreciated.

The TRPC3c isoform is a splice variant in which the complete exon 9 coding region is omitted. Thus, the mouse TRPC3c spliced transcript having a nucleotide sequence set forth in SEQ ID NO:1 (Genbank Acc. No. FJ207476) encodes a TRPC3c protein that has an amino acid sequence set forth in SEQ ID NO:2 (Genbank Acc. No. ACO07350), which lacks amino acids 737 to 764 of the full length mouse TRPC3 protein (TRPC3b) set forth in SEQ ID NO: 4 (Genbank Acc. No. BAC37961) and encoded by the TRPC3b transcript set forth in SEQ ID NO:3 (Genbank Acc. No. AK080619). The rat TRPC3c spliced transcript having a nucleotide sequence set forth in SEQ ID NO:5 (Genbank Acc. No. JN160741) encodes a rat TRPC3c protein that has an amino acid sequence set forth in SEQ ID NO:6 (Genbank Acc. No. AEK22122), which lacks amino acids 739 to 766 of the rat TRPC3b protein set forth in SEQ ID NO:8 (Genbank Acc. No. NP068539) and encoded by the rat TRPC3b transcript set forth in SEQ ID NO:7 (Genbank Acc. No. NM021771). The guinea pig TRPC3c spliced transcript having a nucleotide sequence set forth in SEQ ID NO:5 (Genbank Acc. No. FJ207474) encodes a guinea pig TRPC3c protein that has an amino acid sequence set forth in SEQ ID NO:6 (Genbank Acc. No. ACO07348), which lacks amino acids 737 to 764 of the guinea pig TRPC3b protein set forth in SEQ ID NO:8 (Genbank Acc. No. NP001166502) and encoded by the guinea pig TRPC3b transcript set forth in SEQ ID NO:7 (Genbank Acc. No. NM021771).

In mouse, rat and guinea pig, the TRPC3c spliced transcript lacks 84 bp (corresponding to 28 amino acids) compared to the TRPC3b transcript. There is 100% sequence conservancy of this region at the protein level in all three species and also in human TRPC3, and conservation of the intron-exon boundaries. Accordingly, a human TRPC3c isoform is also likely expressed as a splice variant lacking amino acids encoded by exon 9. Exemplary predicted human TRPC3c polypeptides include, but are not limited to, those having amino acid sequences set forth in SEQ ID NOS:23 and 26.

As disclosed herein for the first time, TRPC3c splice variants confer increased cation flux compared to the full length TRPC3b isoform that was previously thought to be solely expressed in the brain. This larger cation flux in TRPC3c expressing cells is due to increased TRPC3c channel opening frequency, as there appears to be no difference in channel conductance or selectivity between the two isoforms. The TRPC3c channel activity is modulated by cytosolic Ca²⁺, where removal of Ca²⁺ provides maximum activation of the channels, and addition of Ca²⁺ rapidly inhibits channel opening. The enhanced Ca²⁺ entry arising from the increased opening frequency of the TRPC3c channels is associated with a significant increase (e.g. five-fold) increase in cytosolic Ca²⁺ concentration following channel activation, compared with TRPC3b expressing cells. This elevated mGluR1-activated TRPC3 current in cerebellar Purkinje cells can be blocked by a TRPC blocker, such as genistein.

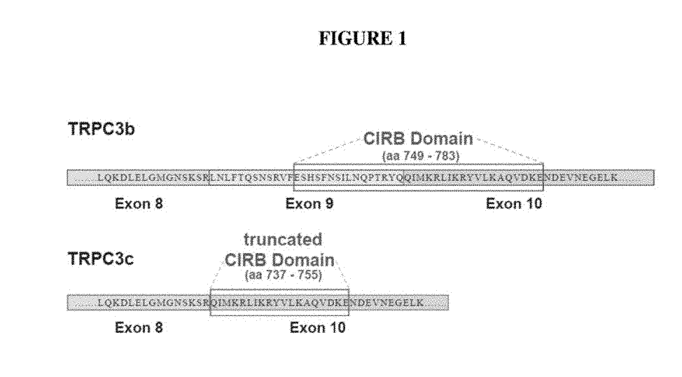

While not being bound by theory, the increased opening rate of the TRPC3c channel observed in the studies described herein is likely to be the result of altered regulation at the CIRB domain, of which a significant portion is missing in TRPC3c variants from alternative splicing to remove exon 9 (FIG. 1). It is likely that TRPC3c channels have reduced affinity for calmodulin binding to the CIRB domain, thereby reducing its inhibitory effect on the channel overall. As shown in Example 3, the TRPC3c isoform exhibits a different sensitivity to Ca²⁺ compared to TRPC3b, with a greater spontaneous opening rate observed with TRPC3c channels exposed to nominal intracellular Ca²⁺, indicating that TRPC3c channels are not efficiently regulated via the CIRB domain.

TRPC3c channels can therefore facilitate significant Ca²⁺ entry and sustained membrane depolarization of neurons, particularly in response to glutamate release through activation of mGluR. As determined herein, the membrane depolarization and Ca²⁺ entry are comparable to that of the NMDA receptor. Unregulated glutamate release, such as that which occurs following stroke, epileptic episodes, head trauma (such as contusions and blunt force trauma), and severe blood loss, can result in sustained depolarization and Ca²⁺ entry associated with activation of the mGluR. These events can result in neuron apoptosis and necrosis, particularly of cerebellar Purkinje cells in which TRPC3c expression is most dominant. Indeed, cerebellar Purkinje cells have been shown to be particularly susceptible to ischaemic injury (Hausmann et al. (2007) Int J Legal Med 121:175-183).

Accordingly, inhibitors of TRPC3c channels can be used in the methods described herein to block these apoptotic and necrotic cell death pathways, thereby inhibiting or preventing brain injury associated with excess glutamate release, such as that observed following stroke, epilepsy, head trauma (such as contusions and blunt force trauma), severe blood loss, cessation of blood flow, cardiac arrest and other ischaemic events.

### TRPC3 Inhibitors

Any agent that inhibits TRPC3c channel formation, activation or activity can be used in the methods and compositions described herein to inhibit or prevent brain injury associated with excess glutamate release. Inhibition of TRPC3c channel formation, activation or activity can be effected by inhibiting TRPC3c expression and/or inhibiting TRPC3c activity (e.g. the ability of TRPC3c to form channels and facilitate cation flux).

TRPC3 inhibitors include small molecules (e.g. chemical entities), proteins, and nucleic acid molecules that block or inhibit TRPC3c channel activation or formation. In some embodiments, the TRPC3 inhibitor is specific for TRPC3 channels. In other embodiments, the TRPC3 inhibitor is a non-specific inhibitor, such as a tyrosine kinase inhibitor, and inhibits the activation or formation of TRPC3c and one or more other TRPC channels, such as TRPC1, TRPC4, TRPC5, TRPC6 or TRPC7. In further embodiments, the inhibitor is specific for TRPC3c, such that TRPC3b and other TRPC channels are unaffected by exposure to the inhibitor.

In some instances, the TRPC3 inhibitors used in the methods and compositions of the present invention can cross the blood brain barrier (BBB) to facilitate efficient delivery of the inhibitor to the TRPC3c-expressing neurons. However, as would be understood by those skilled in the art, this is not necessarily required. For example, the BBB is often compromised in diseases and conditions associated with excess glutamate release, such as stroke, and inhibitors that may not cross the BBB in healthy individuals can do so in individuals suffering brain injury. Specialized delivery methods also can be used to facilitate passage of an inhibitor across the blood brain barrier. Inhibitors can be engineered for receptor-mediated transport across the BBB by, for example, transferrin receptors, insulin receptors and low-density lipoprotein receptors. In such instances, the inhibitor is linked to the endogenous ligands or monoclonal antibodies that bind these receptors to trigger transport across the BBB (see e.g. Pardridge and Boado (2012) Methods Enzymology 503:269-292). Nanocarriers have also been shown to be able to deliver agents across the BBB (see e.g. Bhaskar et al. (2010) Part Fibre Toxicol. 7:3). Methods of temporarily permeabilising the BBB also can be used. For example, administration of an adenosine receptor agonist has been shown to modulate BBB permeability and facilitate delivery of an intravenously injected antibody to the brain (Carman et al. (2012) J Neurosci. 31 (37):13272-80). Other agents, including mannitol and bradykinnin, as well as methods such as focused ultrasound, can also be used to temporarily disrupt the BBB and facilitate delivery of therapeutic agents to the brain (Etame et al. (2012) Neurosurg Focus. 32 (1):E3).

In some embodiments, the TRPC3 inhibitor used in the methods and compositions of the present invention is a small molecule, such as a chemical compound. Exemplary small molecules include, but are not limited to, tyrosine kinase inhibitors such as genistein (4′, 5, 7-trihydroxyisoflavone or 5, 7-dihydroxy-3-(4-hydroxyphenyl)-4H-1-benzopyran-4-one) and PP2 (3-(4-chlorophenyl) 1-(1,1-dimethylethyl)-1H-pyrazolo[3,4-d]pyrimidin-4-amine), 2-aminoethoxydiphenylborane (2-APB), SKF96365 (Guillermo Vazquez et al. (2004) Biochimica et Biophysica Acta 1742:21-36) and bis(trifluoromethyl)pyrazoles (BTPs) such as 4-methyl-4′-[3,5-bis(trifluoromethyl)-1H-pyrazol-1-yl]-1,2,3-thiadiazole-5-carboxanilide (BTP2), ethyl-1-(4-(2,3,3-trichloroacrylamide)phenyl)-5-(trifluoromethyl)-1H-pyrazole-4-carboxylate (Pyr3), norgestimate, erbstatin-analog, herbimycin, lavendustin A (Vazquez G et al. (2004) J Biol Chem 279:40521-40528) and the TRPC3 inhibitors described in Int. Pat. Pub. No. WO2012037349.

In one embodiment, genistein, which has been shown to efficiently cross the BBB, is used in the methods and compositions of the present invention. Genistein is a well-characterized isoflavone found in a number of plants. It is a tyrosine kinase inhibitor that has been shown to inhibit src tyrosine kinase-mediated phosphorylation of TRPC3 (Kawasaki et al. (2006) PNAS 103:335-340) and, as demonstrated below, can block TRPC3c-mediated current in cerebellar Purkinje cells. Any form of genistein can be used in the methods of the present invention providing that form retains the ability to block TRPC3c channel activation or formation. For example, various crystalline forms of genistein can be used, including, but not limited to, crystalline genistein sodium salt dihydrate; crystalline genistein potassium salt dihydrate; crystalline genistein calcium salt; crystalline genistein magnesium salt; crystalline genistein L-lysine salt; crystalline genistein N-methylglucamine salt; crystalline genistein N-ethylglucamine salt; crystalline genistein diethylamine salt; and crystalline genistein monohydrate, as described in U.S Pat. Pub. No. 20120035253.

In another embodiment, a specific TRPC3 inhibitor, such as Pyr3, is used in the methods and compositions of the present invention. Pyr3 (ethyl-1-(4-(2,3,3-trichloroacrylamide)phenyl)-5-(trifluoromethyl)-1H-pyrazole-4-carboxylate) is a pyrazole compound that has been shown to selectively inhibit TRPC3-mediated Ca²⁺ influx, while having no effect of other TRPC channels (Kiyonaka et al. (2009) PNAS 106:5400-5405). Accordingly, Pyr3 or other TRPC3-specific inhibitors can be used in embodiments of the present invention to specifically inhibit TRPC3-mediated Ca²⁺ flux while not interfering with other TRPC channel activity.

In further embodiments, the TRPC3 inhibitor used in the provided methods and compositions is a protein or peptide. For example, antibodies and antigen-binding fragments thereof, such as Fab fragments, F(ab′)₂ fragments, F(ab′)₃ fragments, Fv fragments, disulfide-linked Fvs (dsFv), Fd fragments, Fd′ fragments, single-chain Fvs (scFv), (scFv)₂ single-chain Fabs (scFab), diabodies, triabodies, tetrabodies, anti-idiotypic (anti-Id) antibodies, that bind TRPC3c and block TRPC3c channels activation or formation are suitable for embodiments of the invention. The antibodies or antigen-binding fragments thereof can be specific for (i.e. specifically bind to) TRPC3c such that they do not bind to and inhibit TRPCb or other TRPC channels. In other embodiments, the antibodies or antigen-binding fragments thereof can be specific for TRPC3, such that they specifically bind all TRPC isoforms including TRPC3b and TRPC3c, but do not bind and inhibit other TRPC proteins. In further examples, the antibodies and antigen-binding fragments recognise and bind to all TRPC proteins, including TRPC3c, and inhibit the formations and/or activation of all TRPC channels.

Methods of generating antibodies and antigen-binding fragments specific for a particular protein or epitope are well known in the art and can be used to generated antibodies and antigen-binding fragments that bind to TRPC3c and inhibit TRPC3c channel formation and/or activation. For example, antibodies or antigen-binding fragments thereof can be produced by immunising an animal with TRPC3c. Antibodies and antigen-binding fragments thereof can then be isolated directly from the animal, such as from the plasma, or can be isolated following generation of monoclonal antibodies from hybridomas. In other instances, the antibodies or antigen-binding fragments thereof are produced from antibody libraries, and selected using display and panning methods well known in the art.

The antibodies and antigen-binding fragments can be further modified using methods well known in the art. For example, modifications can be made to increase binding, by for example, affinity maturation, or to decrease immunogenicity by removing predicted MHC class II-binding motifs. Numerous methods for affinity maturation of antibodies are known in the art. Many of these are based on the general strategy of generating panels or libraries of variant proteins by mutagenesis followed by selection and/or screening for improved affinity, such as by selection by panning methods described above. Mutagenesis is often performed at the DNA level, for example by error prone PCR, by gene shuffling, by use of mutagenic chemicals or irradiation, by use of ‘mutator’ strains with error prone replication machinery or by somatic hypermutation approaches that harness natural affinity maturation machinery. Mutagenesis can also be performed at the RNA level, for example by use of Qβ replicase. Library-based methods allowing screening for improved variant antibodies can be based on various display technologies such as phage, yeast, ribosome, bacterial or mammalian cells, and are well known in the art. Affinity maturation can also be achieved by more directed/predictive methods for example by site-directed mutagenesis or gene synthesis guided by findings from 3D protein modelling.

TRPC3 inhibitors for use in the present invention also include inhibitory nucleic acids, such as antisense oligonucleotides, ribozymes, miRNAs and siRNAs, that target TRPC3c transcripts. It is well within the skill of a skilled artisan to design and produce nucleic acid molecules such as antisense oligonucleotides, ribozymes, miRNAs and siRNAs that target TRPC3c transcripts. For example, siRNA molecules that target TRPC3 and inhibit TRPC3 channel formation are known in the art (Lanner et al. (2009) FASEB J 23:1728-1738). In some embodiments, only TRPC3c mRNA and not TRPCb mRNA is targeted by the nucleic acid molecules. In other instances, the nucleic acid molecules recognize and bind to both TRPC3c and TRPC3b, inhibiting the formation of channels with either isoform.

It is well within the skill of those in the art to select an appropriate inhibitor for use in the methods of the present invention. For example, as will be understood by those skilled in the art, apoptosis or necrosis of neurons in acute conditions associated with excess glutamate release, such as stroke or traumatic brain injury, should be inhibited with agents that inhibit an activity of the TRPC3c protein (e.g. the ability of the TRPC3c protein to form channels, the ability of the TRPC3c channels to be activated and facilitate Ca²⁺ flux), thereby immediately inhibiting Ca²⁺ entry into neurons. Conversely, nucleic acids, such as antisense oligonucleotides, ribozymes, miRNAs and siRNAs, that target TRPC3c transcripts can be administered to subjects with chronic conditions, such as epilepsy, where ongoing inhibition of TRPC3c channel formation may be desirable.

The efficacy of the TRPC3 inhibitors can be assessed using methods and assays well known in the art, including, for example, the assays described in the Examples below. For example, in vitro assays can be used to determine the effect of the inhibitor on Ca²⁺ flux in neurons (see e.g. Example 5). In vivo assays using small animal models can be used to assess the effect of the inhibitor on neuroprotection following brain injury, such as brain injury associated with oxygen deprivation.

### Formulations and Administration of TRPC3 Inhibitors

TRPC3 inhibitors can be formulated for in vitro or in vivo use. For example, in some aspects, the TRPC3c inhibitors are formulated for in vitro use, such as in vitro assays in which neurons are exposed to a TRPC3c inhibitor. In other examples, TRPC3 inhibitors are formulated for in vivo use, such as for administration to a subject.

In particular embodiments of the present invention, TRPC3 inhibitors are formulated as pharmaceutical compositions and administered to a subject suffering from a glutamate-associated disease or condition, such as stroke, epilepsy, severe blood loss and/or head trauma (such as a contusion or blunt force trauma, or other ischaemic events), to inhibit necrosis or apoptosis of neurons. Apoptosis or necrosis of neurons can be inhibited in any region of the brain, including, but not limited to, the cerebellum, the midbrain, the cerebrum and/or the medulla. In particular embodiments, apoptosis and/or necrosis of Purkinje cells in the cerebellum is inhibited by administration of a composition comprising a TRPC3 inhibitor.

Generally, compositions containing a TRPC3 inhibitor are prepared in view of approval from a regulatory agency or otherwise prepared in accordance with generally recognized pharmacopeia for use in animals and in humans. Compositions can contain, in addition to the TRPC3 inhibitor, a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A pharmaceutical composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

The compositions can be formulated for administration by any route. The most appropriate route of administration can be determined by a person of skill in the art, taking into account the particular disease or condition being treated. For example, the compositions comprising a TRPC3 inhibitor can be formulated for parenteral, intravenous, intraarterial, subcutaneous, intramuscular, intracranial, intraorbital, ophthalmic, intraventricular, intranasal, or oral administration. In some embodiments, therapeutic formulations comprising TRPC3c are in the form of liquid solutions or suspensions for intravenous administration. Also encompassed by the present invention are formulations for controlled release of a TRPC3 inhibitor.

The compositions comprising a TRPC3 inhibitor are formulated with an amount or concentration of a TRPC3 inhibitor that is suitable for the embodiments of the present invention, i.e. at concentrations or amounts sufficient to inhibit the necrosis and/or apoptosis of neurons when administered to a subject. The compositions can be formulated for direct administration to a subject, or can be formulated as a concentrated composition that is subsequently diluted prior to use. In particular embodiments, the compositions are in liquid form and are formulated with between about 1 ng/mL to about 100 mg/mL of a TRPC3 inhibitor, between about 10 ng/mL and about 10 mg/mL, between about 100 ng/mL and about 10 mg/mL, between about 1 μg/mL and about 10 mg/mL, between about 10 μg/mL and about 1 mg/mL, or between about 100 μg/mL and about 1 mg/mL of TRPC3 inhibitor. In some instances, the compositions are in solid form, such as in tablet or capsule form, and contain the TRPC3 inhibitor at between about 0.001% (w/w) to about 50% (w/w), between about 0.01% (w/w) to about 20% (w/w), between about 0.5% (w/w) to about 10% (w/w), or between about 1% (w/w) to about 5% (w/w). The most suitable concentration to achieve the desired effect will depend on a number of factors and may be determined by those skilled in the art using routine experimentation.

The compositions comprising a TRPC3 inhibitor can include one or more TRPC3 inhibitors, including 2, 3, 4, 5, or more TRPC3 inhibitors. The compositions comprising a TRPC3 inhibitor can also contain one or more additional active agents. For example, the TRPC3 inhibitor compositions provided herein can include one or more other active agents useful in the treatment or stabilization of subjects suffering from stroke, epilepsy, severe blood loss and/or head trauma. Non-limiting examples of active agents that can be included in the compositions provided herein include other neuroprotective agents, thrombolytic agents (e.g. tissue plasminogen activator), insulin, antiplatelet agents (e.g. aspirin, clopidogrel and dipyridamole), anticoagulants (e.g. heparin, warfarin and dabigatran), anticonvulsant agents (e.g. divalproex sodium (valproic acid), lithium carbonate, lamotrigine, lithium citrate, lithium carbonate, gabapentin, carbamazepine, topiramate and oxcarbazepine), procoagulants (e.g. Factor VIIa).

The formulations can be administered to a subject in therapeutically effective amounts, e.g., amounts that inhibit the apoptosis or necrosis of neurons. The precise amount or dose of the TRPC3 inhibitor that is administered to the subject depends on several factors, including, but not limited to, the activity of the inhibitor, the use of other therapeutic agents, the route of administration, the number of dosages administered, and other considerations, such as the weight, age and general state of the subject. Particular dosages can be empirically determined or extrapolated from, for example, studies in animal models or previous studies in humans.

The compositions, including pharmaceutical compositions, containing a TRPC3 inhibitor can be administered by any method and route understood to be suitable by a skilled artisan, including, but not limited to, intravenous (including by discrete injection, intravenous bolus or continuous infusion), intramuscular, intradermal, transdermal, parenteral, intracranial, intraarterial, intraorbital, subcutaneous, intranasal, oral, intraperitoneal or topical administration, as well as by any combination of any two or more thereof, formulated in a manner suitable for each route of administration.

In the methods provided herein, a composition comprising a TRPC3 inhibitor is administered to a subject before, during and/or after the subject has experienced an event that results in excess glutamate release in the brain. Exemplary of such events are strokes, epileptic seizures, head trauma, severe blood loss, cardiac arrest and other ischaemic events. A composition comprising a TRPC3 inhibitor can be administered to a subject at any time after the subject has experienced an event that results in excess glutamate release, such as 1 minute, 5 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, 18 hours, 24 hours, 3 days, 4 days, 5 days, 6 days, 7 days, 2 weeks, 3 weeks, 4 weeks or more after the subject has experienced an event that results in excess glutamate release in the brain. The TRPC3 inhibitor can be administered once or more than once, such 2, 3, 4, 5, 6 or more times.

In some embodiments of the present invention, a TRPC3 inhibitor is administered to a subject in combination with one or more other therapies, including surgical therapies and therapies involving the administration of one or more other therapeutic agents, such as another neuroprotective agent, a thrombolytic agent (e.g. tissue plasminogen activator), an insulin, an antiplatelet agent (e.g. aspirin, clopidogrel and dipyridamole), an anticoagulant (e.g. heparin, warfarin and dabigatran), an anticonvulsant agent (e.g. divalproex sodium (valproic acid), lithium carbonate, lamotrigine, lithium citrate, lithium carbonate, gabapentin, carbamazepine, topiramate and oxcarbazepine), and/or a procoagulant (e.g. Factor VIIa). For example, a subject suffering a stroke can be administered a TRPC3 inhibitor and another neuroprotective agent, a thrombolytic agent, an insulin and/or an anticoagulant. A subject suffering an epileptic seizure can be administered a TRPC3 inhibitor and an anticonvulsant agent. A subject suffering severe blood loss can be administered a TRPC3 inhibitor and procoagulant. In such instances, the TRPC3 inhibitor can be administered simultaneously and/or sequentially to the other therapy. For example, the TRPC3 inhibitor can be administered to the subject at the same time, before and/or after a surgical procedure is performed on the subject. Similarly, the TRPC3 inhibitor can be administered to the subject at the same time, before and/or after another therapeutic is administered to the subject. In embodiments where a subject is administered a TRPC3 inhibitor and one or more other therapeutic agents, the TRPC3 inhibitor and the one or more other therapeutic agents can be in the same or different compositions, and can be administered by the same or different routes.

In particular embodiments of the present invention, a subject that has experienced or is experiencing a stroke is administered a TRPC3 inhibitor and a thrombolytic agent, such as tissue plasminogen activator. The TRPC3 inhibitor and the thrombolytic agent can be administered simultaneously or sequentially. In particular embodiments, the TRPC3 inhibitor is administered to the subject after administration of the thrombolytic agent. Typically, the thrombolytic agent is administered to the subject as soon as the subject is identified as having suffered as stroke, provided it is within 1, 2, 3, 4 or 5 hours of the subject experiencing the stroke, more typically within 3 hours of the stroke. The TRPC3 inhibitor can be administered at the same time as the thrombolytic agent or after, such as 1 hour, 2 hours, 3 hours, 4 hours, 5 hours, 6 hours, 7 hours, 8 hours, 9 hours, 10 hours, 11 hours, 12 hours, 18 hours, 24 hours, 3 days, 4 days, 5 days, 6 days, 7 days, 2 weeks, 3 weeks, 4 weeks or more after the thrombolytic agent has been administered.

In other embodiments of the present invention, the TRPC3c inhibitors are exposed to cells in vitro, such as in assays to assess TRPC3c inhibitor specificity and/or activity. The cells can be neurons or other cells, such as cells expressing recombinant TRPC3c. In particular aspects, the cells exposed to a TRPC3c inhibitor are also exposed to an activating agent that activates TRPC3c channels. Accordingly, also provided herein are methods in which a cell is contacted with or exposed to a TRPC3 inhibitor. Typically, various parameters are then assessed, such as membrane conductance and cation flux.

Those skilled in the art will appreciate that the aspects and embodiments described herein are susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The citation of any reference herein should not be construed as an admission that such reference is available as “Prior Art” to the present application. Further, the reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure is further described by reference to the following non-limiting examples.

### Example 1. Characterization of TRPC3 and TRPC3 Expression in Brain Tissue

To characterize TRPC3 gene transcription in brain tissue, in particular the relative expression of the full length TRPC3 isoform (TRPC3b) and the TRPC3 isoform that lacks exon 9 (TRPC3c), reverse transcription and PCR amplification was performed from RNA extracted from mouse, rat and guinea pig brain tissues (cerebellum, midbrain, medulla and cerebrum). For mouse (C57BL/6J strain) and rat (wistar) brain tissues, the total RNA was extracted using Trizol (Invitrogen, U.S.A.) according to the manufacturer's instruction. For guinea pig tissues, total RNA was extracted using Purelink total RNA isolation kit (Invitrogen, U.S.A.). The number of animals for each brain region in these experiments were as follows: mouse: n=9 cerebellum, n=5 mid-brain, medulla, n=4 cerebrum; rat n=6 cerebellum, mid-brain, medulla, n=5 cerebrum.

The RNA was then reverse transcribed using Superscript III Reverse Transcription System (Invitrogen, U.S.A.), with random hexamer priming, to produce first-strand cDNA template.

The TRPC3 cDNA was amplified using primers that span exon 9 in order to investigate the relative expression of each isoform. The PCR amplification (40 cycles) used forward and reverse primers that targeted the coding regions of exon 8 and 10 of TRPC3 mRNA, respectively; denaturation at 98° for 10 seconds C, annealing at 58° C. for 15 seconds and extension at 72° C. for 30 seconds. The sequence of the primers and size of the resulting amplicons of both isoforms in base pairs were as follows:

mouse:
(SEQ ID NO: 13)
forward 5′-CTAACTTTTCCAAATGCAGGAGGAGAAG-3′;
(SEQ ID NO: 14)
reverse 5′-TCGCATGATAAAGGTAGGGAACACTAGA-3′;
generating a TRPC3b amplicon of 501 nucleotides
(nt) and TRPC3c amplicon of 417 nt.
rat:
(SEQ ID NO: 15)
forward 5′-CAGTGATGTAGAGTGGAAGTTTGC-3′;
(SEQ ID NO: 16)
reverse 5′-CTCCCTCATTCACACCTCAGC-3′;
generating a TRPC3b amplicon of 408 nt and TRPC3c
amplicon of 324 nt.
guinea-pig:
(SEQ ID NO: 17)
forward 5′-GGATCATTAACTTTTCCAAATGTAGAAGG-3′;
(SEQ ID NO: 17)
reverse 5′-TCTCAGCACGCTGGGATTCAGTTTCT-3′;
generating a TRPC3b amplicon of 374 nt and TRPC3c
amplicon of 290 nt.

The amplicons resulting from the PCR were then separated using electrophoresis on 1% agarose gel. The cDNA of each TRPC3 isoform was quantified via measurement of the optical density of the respective bands (SYBR-Safe™ stained; Invitrogen, U.S.A.) using semi-quantitative analysis software Genesnap (v6.08, Perkin Elmer, U.S.A.).

Immunofluorescence and confocal microscopy using an anti-TRPC3 antibody was then used to confirm expression of TRPC3 in the mouse brain. Mice (C129 SvEv background strain) were euthanized with sodium pentobarbital solution (100 mg/ml; 100 mg/kg body weight), intracardially perfused with 10 ml of 0.5% sodium nitroprusside in 0.9% saline, followed by perfusion with 20 ml of 4% paraformaldehyde (PFA) in 0.1 M phosphate buffer. Cerebellum was then dissected and post-fixed in the PFA solution overnight. The tissue was then cryoprotected (10%, 20%, 30% sucrose in PBS), embedded in Tissue-Tek® O.C.T. compound (Sakura Finetek, U.S.A.) and sectioned at 50 μm using a cryostat (floating sections). Brain sections were then permeabilized with 1% Triton X-100 in PBS with 10% normal goat serum (NGS, Vector Laboratories, U.S.A.) for 2 hours for cerebellum sections at room temperature, followed by overnight incubation with TRPC3 antibody (1:1000; lot nos. AN-03 or AN-07; Alomone, Israel) in PBS with 5% NGS and 0.1% Triton X100, at 4° C. After washing in PBS (3×30 min), Alexa Fluor® 488 goat anti-rabbit IgG secondary antibody (Invitrogen, U.S.A.; 1:500, 5% normal goat serum, 0.1% Triton X-100, PBS) was applied for 4 h at room temperature, followed by 2 h at 4° C., during which the tissue was protected from light. The floating cerebellar sections were then washed in PBS several times and mounted using Vectashield® (Vector Laboratories, U.S.A.). The immunolabeling was then visualised using a Zeiss D1 AxioExaminer NLO710 confocal microscope with 40× objective, 488 nm excitation laser (495-550 nm emission).

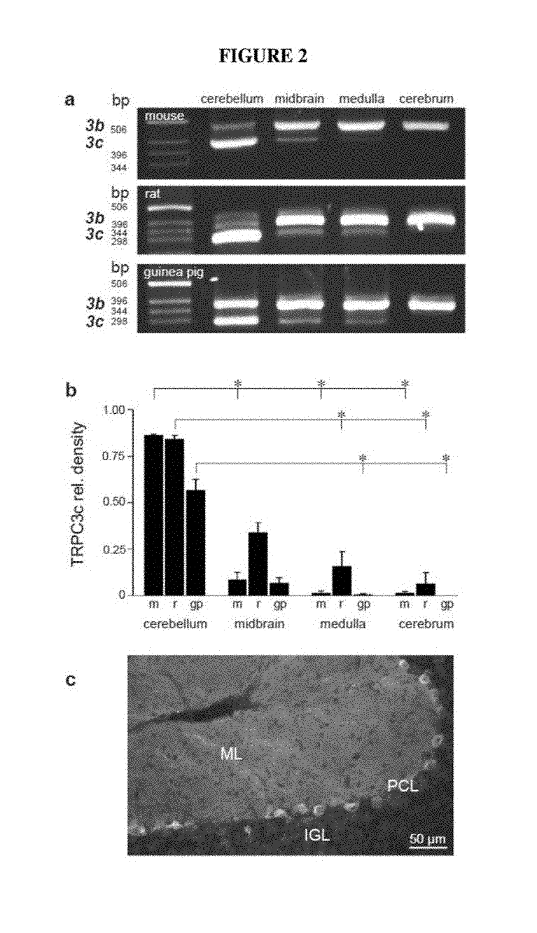

As shown in FIG. 2A, there is brain region-dependent alternative splicing of TRPC3 mRNA in mouse, rat and guinea pig. In all three species, the cerebellum showed dominant expression of the isoform designated TRPC3c, which was smaller than the TRPC3b (unspliced) isoform by 84 bp as determined by sequencing of cloned cDNA. The proportion of TRPC3c relative to TRPC3b in different brain regions (cerebellum, midbrain, medulla and cerebral cortex) was compared by optical density measurement of agarose gel electrophoresis images (FIG. 2B). ANOVA for these data for each species indicated that there were significant differences (mouse, p<0.001; Rat, p<0.001; guinea-pig, p<0.001) in the proportion of TRPC3c:TRPC3b transcript across brain regions. In both mouse and rat cerebellum, the TRPC3c isoform comprised more than 80% of the cDNA amplicon; with guinea-pig cerebellar tissue exhibiting approximately equivalent levels of TRPC3c and TRPC3b expression. The TRPC3c isoform was also detectable in the other brain regions, with the lowest relative level in each species found in the cerebral cortex.

Immunofluorescence indicated that the localization of TRPC3 in the mouse cerebellum was largely confined to the Purkinje neurons (FIG. 2C), as previously described (Huang et al., (2007) Cell Calcium 42:1-10; Hartmann et al., (2008) Neuron 59:392-398). Given the semi-quantitative mRNA expression data, it was concluded that the TRPC3c isoform contributes the majority of the TRPC3 immunoreactivity present in Purkinje cells.

For the mouse cDNA template, primers spanning the full coding region were used to exclude additional alternative splicing.

Animal experiments were undertaken with protocol approval of the University of New South Wales (Australia) and University of Auckland (New Zealand) animal ethics committees.

### Example 2. Expression of Recombinant Mouse TRPC3b and TRPC3c Channels in HEK293 Cells

The expression of recombinant TRPC3b and TRPC3c channels in stably-transfected human embryonic kidney (HEK) 293 cells was assessed by Western blotting and microscopy.

Full length mouse TRPC3 transcripts were obtained by PCR using similar thermal cycling parameters as those described above. Full length TRPC3b and TRPC3c transcripts were detected by RT-PCR from cerebrum and cerebellum of mouse, respectively, using 5′ sense and 3′ antisense primers that targeted the regions of the start and stop codons. The forward primer had a sequences of 5′-ACAGAATTCCTGCGGGGATGCGTGACA-3′ (SEQ ID NO:19) and the reverse primer had a sequence of 5′-AGCGGATCCCCTCACTCACATCTCAGCA-3′ (SEQ ID NO:20. The restriction sites for EcoR1 and BamH1 were incorporated into the 5′ end of the forward and reverse primers, respectively, to facilitate cloning into the pIRES-DsRed2 mammalian expression vector (Clontech, U.S.A.). All PCR reactions utilized Finnzyme™ high fidelity Taq DNA polymerase and supplied reaction mix (Thermo Scientific, U.S.A.). The resulting TRPC3b and TRPCC3b cDNA sequences were then cloned into the pIRES-DsRed2 mammalian expression vector.

HEK 293 cells (Invitrogen, U.S.A.) were cultured to 90% confluence in Dulbecco's modified eagle medium (DMEM; Invitrogen, U.S.A.) supplemented with 10% fetal bovine serum in a humidified atmosphere of 95% O₂ and 5% CO₂ at 37° C. Cells were transfected with either mouse TRPC3b or TRPC3c cDNA cloned into a pIRES-DsRed2 vector construct (Clontech, U.S.A.). The transfection of the HEK293 cells with the vectors was made using Lipofectamine 2000 (Invitrogen, U.S.A.), according to manufacturer's instructions. Cells were then treated with 1.1 mg/ml of G418 antibiotic (Invitrogen, U.S.A.) for 4 weeks to select for stably transfected cells. The expression of the mouse TRPC3 gene construct in the G418 resistant cells was verified by RT-PCR amplification and sequencing of the TRPC3 cDNA.

The stably transfected cell lines were sorted using FACS for high DsRed2 fluorescence. To do this, cells were trypsinized and sorted using FACSaria™ (BD Bioscience, U.S.A.) cell sorting apparatus, which selected for DsRed2 signal using a PE-A filter. Cells with top 5% level of DsRed2 fluorescence were selected for experiments. Co-expression of mGluR1 with the TRPC3 isoforms was facilitated with the use of a mouse mGluR1-eYFP fusion protein encoding cDNA construct downstream of the CMV promoter (Masu et al., (1991) Nature 349 (6312):760-5). The HEK293 cells stably expressing either TRPC3 isoform were then transformed with the mGluR1-eYFP fusion protein cDNA using Lipofectamine 2000 (Invitrogen, U.S.A).

### Western Blotting of Cell Lysates

Expression of TRPC3b and TRPC3c proteins was quantified in transfected HEK293 cells by Western blotting. HEK293 cells stably expressing TRPC3b or TRPC3c, and untransfected cells (control), were grown in minimum essential medium containing 10% fetal bovine serum, streptomycin, and penicillin and prior to collection, were plated out overnight at a density of 1.5×10⁵ cells/well in poly-D-lysine coated 6-well (6.9 cm² area) culture dishes at 37° C. with 5% CO₂.

Whole-cell lysates were prepared by incubating cells in lysis buffer (137 mM NaCl, 20 mM Tris, 1 mM EDTA, 1% Triton X-100, 1% sodium deoxycholaste, 1% SDS, 0.1% protease inhibitors (Complete™ Mini protease inhibitor mixture; Roche Applied Sciences, U.S.A.) adjusted to pH 7.5 with HCl), for 30 minutes at room temperature with agitation, insoluble content was removed by centrifugation. Cell lysates were separated by 10% SDS-polyacrylamide gel electrophoresis (SDS-PAGE) at 10 μg/lane in 2× Lammeli sample buffer (125 mM Tris, 4% SDS, 20% glycerol, and 10% β-mercaptoethanol).

TRPC3b and TRPC3c proteins were detected by Western blotting using polyclonal rabbit antibody directed to amino acids 822-835 of mouse TRPC3 at 2 μg/ml (ACC-016, lot no. AN-07; Alomone Labs Ltd, Israel) with a goat anti-rabbit IgG-HRP conjugate secondary antibody (1:20,000, lot no. L9704446 RevA; Bio-Rad, U.S.A.). Chemiluminescence was detected using enhanced chemiluminescence reagent (ECL, Bio-Rad, U.S.A.) and a ChemiDoc digital imaging system (Bio-Rad). To confirm equal protein loading of the whole-cell lysate samples, blots were stripped of TRPC3 antibodies and actin expression level was detected with a rabbit anti-actin affinity isolated antibody (1:1,000, lot no. 048k4861; Sigma-Aldrich, U.S.A.).

### Western Blotting of Plasma Membrane Fraction

Integration of TRPC3 channel subunits into the plasma membrane was determined by Western blotting following extraction of the plasma membrane fraction using membrane-impermeant biotinylation reagent (EZ-Link Sulfo-NHS-SS-Biotin, Pierce Biotechnology, U.S.A.). Cells were washed three times with 2 ml ice-cold phosphate buffered saline (PBS), incubated with 0.5 ml of 1.5 mg/ml sulfo-NHS-SS-Biotin in PBS for 20 min at 4° C., removed by aspiration, and repeated with a fresh aliquot of sulfo-NHS-SS biotin. Unbound biotin was removed by aspiration and cells thoroughly washed and quenched with ice-cold PBS with 100 mM glycine on ice before solubilised in lysis buffer by gentle agitation on ice for 30 min. Cell lysates were collected by centrifugation. To isolate biotinylated cell surface protein, NeutrAvidin™ beads (Pierce Biotechnology. U.S.A) prepared as 50% suspension in lysis buffer were added at 50 μl to 0.19 ml of each lysate supernatant and incubated for 1 hour at 4° C. with occasional mixing. NeutrAvidin beads-biotinylated membrane protein complex was pelleted by centrifugation at 4° C. The unbound supernatant fraction was removed, the pellet washed three times with 0.5 ml lysis buffer, and the biotinylated proteins were extracted from the beads by adding 50 μl 2× Lammeli sample buffer and incubated for 30 min at room temperature for one hour followed by centrifugation. Biotinylated proteins were analysed by SDS-PAGE and Western blotting. Purity of the isolated cell surface protein sample was confirmed by nil expression of actin (data not shown).

### Confocal Immunofluorescence

Localisation of TRPC3 protein expression in HEK293 cells expressing the recombinant mouse TRPC3b and TRPC3c variants was observed by immunofluorescence and confocal microscopy. HEK293 cells grown on poly-D-lysine (Sigma-Aldrich, U.S.A.) coated coverslips, were fixed in situ using 4% PFA in PBS for 10 min, then the cells were washed with PBS. The cells were then permeabilized with 1% Triton X-100 in PBS with 10% normal goat serum (NGS, Vector Laboratories, U.S.A.) for 10 minutes at room temperature, followed by overnight incubation with TRPC3 antibody (1:1000; lot nos. AN-03 or AN-07; Alomone, Israel) in PBS with 5% NGS and 0.1% Triton X100, at 4° C. After washing in PBS (3×30 min), Alexa Fluor® 488 goat anti-rabbit IgG secondary antibody (Invitrogen, U.S.A.; 1:500, 5% normal goat serum, 0.1% Triton X-100, PBS) was applied for 4 hours at room temperature, followed by 2 hours at 4° C., during which the cells were protected from light. The coverslips with the HEK293 cells were then washed in PBS several times and mounted using Vectashield® (Vector Laboratories, U.S.A.). The immunolabeling was then visualised using a Zeiss AxioExaminer FS 710 NLO confocal microscope with 40× objective, 488 nm excitation laser (495-550 nm emission). Controls included incubation without the primary antibody (to assess non-specific secondary binding) and use of untransfected HEK293 cells.

### Results

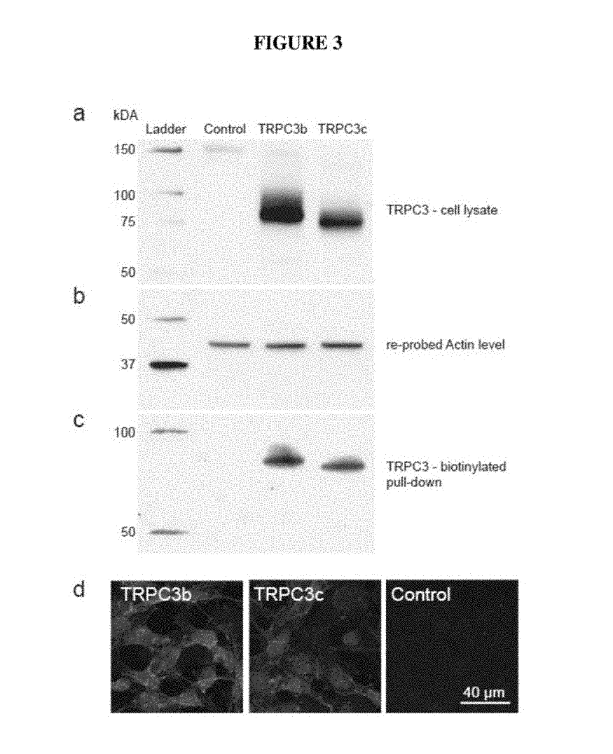

Channel specific protein species were detected at ˜75 kDa (FIG. 3A). The TRPC3c isoform migrated slightly further, consistent with the small size predicted from the loss of the 28 amino acids encoded by exon 9. TRPC3 expression was not detected in whole cell lysates from untransfected HEK293 cells. HEK293-TRPC3c protein levels appeared somewhat less than TRPC3b, most likely due to differences in copy numbers. Equal protein loading was confirmed after stripping and reprobing for β-actin (FIG. 3B). Treatment of transfected and untransfected HEK293 cells with sulfo-HNS-SS-biotin followed by purification of biotinylated proteins on NeutrAvidin beads confirmed expression of TRPC3 channels in the plasma membrane of transfected cells only. Consistent with the whole-cell lysates, the biotinylated (cell surface) TRPC3c protein migrated further, given its slightly smaller molecular weight; with equivalent expression level to TRPC3b (FIG. 3C). Trafficking of both TRPC3 isoforms to the plasma membrane was evident with confocal immunofluorescence (FIG. 3D).

### Example 3. Membrane Conductance of TRPC3c

The membrane conductance of the TRPC3 isoforms was assessed using whole cell electrophysiology and single cell channel electrophysiology assays.

### Whole Cell Electrophysiology

For whole cell patch clamp recordings, HEK293 cells stably expressing recombinant TRPC3 ion channels were grown to 85-95% confluence on a coverslip coated with poly-D-lysine and collagen (both from Sigma-Aldrich, U.S.A.). Recording pipettes were made from borosilicate glass (GC120TF-10, Harvard Apparatus, U.K.). The pipette resistance was at 3-6 MΩ (PC-10, Narishige, Japan). The internal solution had the composition: 130 mM CsCl, 2 mM MgCl₂, 10 mM EGTA, 0.3 mM ATP, 0.03 mM GTP, pH at 7.3 adjusted with CsOH. Cells on the coverslip were placed in a microchamber on an inverted microscope (Leica DMIL, Germany) and superfused with HEPES-buffered physiological salt solution (HPSS) containing 120 mM NaCl, 5.4 mM KCl, 2 mM CaCl₂, 1.13 mM MgCl₂, 10 mM glucose, 20 mM HEPES (pH 7.4) at room temperature.

Whole cell patch clamp recordings were made following a gigaseal, using an Axopatch 200 patch clamp amplifier (Molecular Devices, U.S.A.) controlled by software (pClamp 10.2, Molecular Devices, U.S.A.). Cell capacitance was cancelled and series resistance was compensated by ˜90%. Holding voltage was −40 mV, with a voltage ramp (−100 to +50 mV) over 1 second in every 5 seconds of recording to determine basal and TRPC3 channel mediated membrane conductance. Carbachol (used to activate TRPC3 channels via the endogenously expressed M3 AChR) was purchased from Sigma-Aldrich (U.S.A.). DHPG (—(S)-2-amino-2-(3,5-dihydroxyphenyl)acetic acid), a group I mGluR agonist (Schoepp et al. 1994), was purchased from Tocris Bioscience (U.K.). All experiments were undertaken at room temperature.

### Single Channel Electrophysiology

Single channel activity was recorded using cell attached and inside-out patch clamp configurations in HEK293 cells stably expressing the recombinant mouse TRPC3 channels. The bath solution consisted of HPSS solution. Ca²⁺-free HPSS was identical to the normal HPSS except it consisted of 10 mM EGTA with additional 2 mM MgCl₂ substituting for Ca²⁺ (reducing free [Ca²⁺] to <10 nM). Membrane patch recordings were made using a pipette potential of +100 mV (i.e. a holding potential of −100 mV in an excised patch). The single channel recording was made using an Axopatch 200 patch clamp amplifier (Molecular Devices, U.S.A.) controlled by software pClamp 10.2 (Molecular Devices, U.S.A.). Sampling rate was at 125 kHz and the low-pass filter frequency was 5 kHz. Single channel data were analysed using Clampfit 10.2 (Molecular Devices, U.S.A.). The frequency of the channel opening was analysed using a threshold crossing function. The value for threshold was set as 7× standard deviation of the stable baseline (>15 seconds in continuous length). Channel opening frequency was quantified for 30 second epochs around 1 minute after the CCh (100 μM) application. Each transient with an amplitude greater than the set threshold was counted as a single channel opening event. To estimate single channel conductance (from current amplitude), ˜100 opening events were analysed from each of a series of voltage-clamp recordings in inside-out patches in Ca²⁺ free HPSS solution. These data were parsed using a 50 Hz band filter and then detected using the threshold search function of Clampfit. Peak amplitude was measured for opening events that exceeded a threshold set 4.5 pA above the noise floor and showed no evidence of multi-channel activation.

### Results

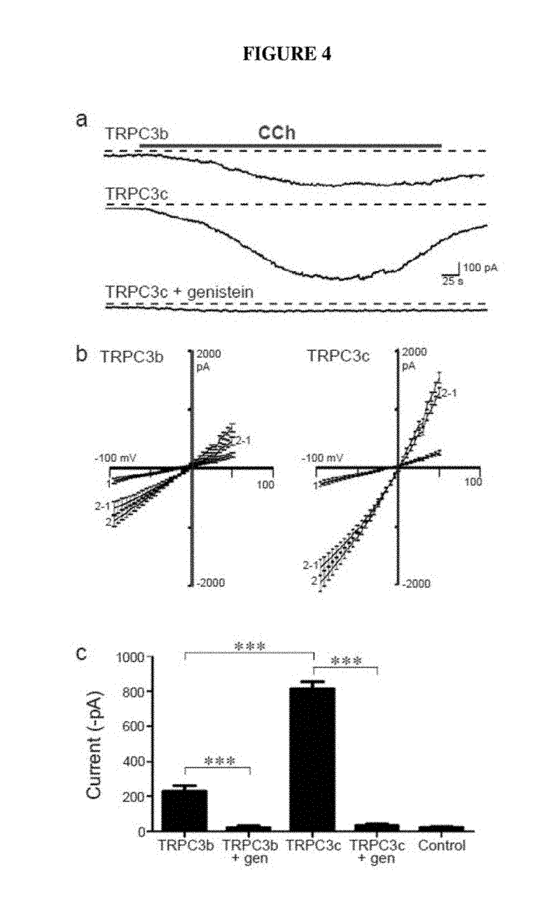

Whole-cell recordings demonstrated slowly activating sustained inward currents with CCh, which were significantly greater in the TRPC3c expressing cells (FIG. 4). The mean peak CCh-activated inward current for TRPC3b versus TRPC3c expressing cells was −235.0±28.7 pA and −809.4±36.9 pA, respectively, at the holding potential Vh=−50 mV (s.e.m.; n=25 and n=29; p<0.001; unpaired t-test). Voltage-ramps confirmed an increased slope conductance with CCh activation that was significantly greater in the TRPC3c expressing cells (CCh increased TRPC3b from 2.6±0.6 nS to 9.9±1.2 nS, n=8; TRPC3c slope conductance increased from 2.8±0.5 nS to 20.4±1.7 nS, n=14; measured about −50 mV; p<0.001 t-test). The corresponding right shifts in zero-current potential (Vz) evident in the current/voltage relationships (I/V, FIG. 4B) changed from −16.9±3.5 mV to −5.6±1.1 mV and −13.9±3.6 mV to −0.4±1.0 mV for CCh-treated HEK293 cells expressing TRPC3b and TRPC3c respectively). There were no significant differences in the reversal potentials (Erev) of the isolated TRPC3 conductances (determined by subtracting the I/Vs obtained during activation by CCh, with the respective I/Vs at rest (Erev(TRPC3b)=−1.9±1.9 mV, n=8; Erev(TRPC3c)=1.1±1.5 mV, n=14; p=0.241, t-test) (FIG. 4B); indicating that the ion selectivities of the two isoforms were similar. Despite the larger TRPC3c currents, TRPC3c expression by the HEK293 cells was weaker than the TRPC3b expression, as determined directly by Western blot (FIG. 3) and also by fluorescence-activated cell sorting (FACS) of isolated cells using the DsRed2 reporter fluorescence, where the mean fluorescence for TRPC3b was 20.7±0.2, n=17024, and for TRPC3c was 11.6±0.2, n=9149 (p<0.001, t-test). CCh-activated current was not observed in untransfected cells (mean=−25.4±4.2 pA; n=7; Vh=−50 mV). In addition, both TRPC3 isoform current responses could be reliably inhibited by genistein, which is a tyrosine kinase inhibitor (200 μM; TRPC3b+CCh+genistein=−24.8±9.7 pA, n=6; TRPC3c+CCh+genistein=−35.4±8.1 pA, n=7; FIG. 4C); P<0.001 for block of each of the isoforms (two way ANOVA with Holm-Sidak post-doc comparisons).

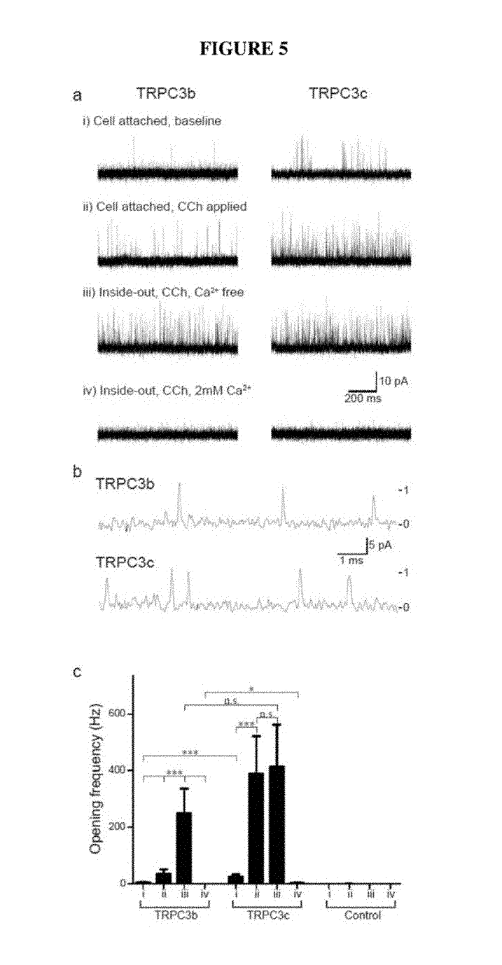

TRPC3b and TRPC3c single channel currents were recorded at a pipette voltage of −100 mV (Vh=+100 mV), with four patch clamp conditions as shown in FIG. 5A(i-iv). Each recording started with measurement of the baseline (i), then activation by CCh (ii) in cell-attached configuration. The patch was then excised, to form an inside-out patch, with the intracellular side of the patch exposed to Ca²⁺ free solution (iii), and finally the patch was exposed to 2 mM Ca2+ (iv). The channel activity featured very brief transients (often below 50 μs), that limited the analysis of channel kinetics. FIG. 5B provides detail of the transient TRPC3 channel opening events. Single channel opening events from inside-out patches in Ca²⁺ free solution of >100 μs duration were analysed using threshold crossing discrimination to estimate current amplitude (TRPC3b and TRPC3c; 7.7±0.21 pA (n=9); and 8.0±0.36 pA (n=5) respectively, p=0.93, t-test). Given the +100 mV holding potential, this provides an estimate of single channel conductance of about 80 pS for both isoforms. These features are consistent with previous characterisation of TRPC3 channels (Zitt et al., (1997) J Cell Biol 138:1333-1341; Zhang et al., (2001) PNAS 98:3168-3173).

Opening frequency was compared for the baseline condition and following activation by CCh (FIG. 5C), with statistical analysis by non-parametric ranked two way ANOVA with Holm-Sidak post-hoc multiple pair-wise comparisons (alpha=0.05). In the cell-attached patch configuration, baseline channel opening frequency was greater for the TRPC3c isoform, (25.2±8.3 Hz, n=16) than that of the TRPC3b isoform (4.4±1.5 Hz, n=19; p<0.001; FIGS. 5 A, i and B, i). Addition of CCh to the bath caused an increase in channel opening activity that was ˜10 fold greater in the TRPC3c isoform compared with TRPC3b (318.3±116.9 Hz, n=16; 34.6±16.3, n=19 respectively; p<0.001) (FIGS. 5 A, ii and B, ii).

In both TRPC3 isoforms, the subsequent excision and exposure of the intracellular side of the patch to the Ca²⁺ free solution elicited a high frequency of channel opening (FIG. 5 A, iii and B, iii). In TRPC3b channels, this was a significant increase (to 248.0±88.0 Hz, n=9) from the cell-attached, CCh-activated state (p<0.001). In contrast, for TRPC3c channels there was no significant difference above the already high opening rate after CCh activation (Ca²⁺ free inside-out patch, 411.9±149.1 Hz; n=5; p=0.43). The apparent maximum activation via the Ca²⁺ free inside-out patch recordings between the TRPC3b and TRPC3c were not significantly different (p=0.238). Finally, exposure of the intracellular side of the inside-out patch to 2 mM Ca²⁺ rapidly reduced channel opening in both TRPC3b isoforms, although the TRPC3c isoform retained a small residual opening rate (TRPC3b, 0.1±0.9 Hz, n=10; TRPC3c, 2.9±1.2 Hz, n=10); p=0.019. The mean opening frequency of untransfected cells was 0.2±0.2 Hz for baseline and 0.3±0.1 Hz for CCh activation (cell attached patch) (n=6). There was no significant difference between baseline and CCh treatment (p=0.694; paired t-test).

### Example 4. Assessment of Ca²⁺ Entry Via TRPC3c Channels

The TRPC3-mediated Ca²⁺ entry through two distinct activation pathways was assessed by microfluorometric Ca²⁺ imaging: i) Ca²⁺ entry via the M3 receptor-PLCB-DAG pathway endogenous to HEK293 cells, using Indo-1 as a Ca²⁺ indicator; and ii) Ca²⁺ entry via mGluR1-PLCB-DAG through co-expression of the mGluR1, using Fluo-4 as a Ca²⁺ indicator.

### Indo-1 and Fluo-4 Microfluorometric Ca²⁺ Imaging

Cells were grown in DMEM media on 18 mm circular coverslips coated with poly-D-lysine (25 μg/ml) and collagen (25 μg/ml) at 1:1 ratio. The cells were washed with HPSS. The coverslips were then incubated with HPSS and 0.1% pluronic acid, along with either 1 μM Indo-1AM or Fluo-4AM Ca²⁺ indicator (Invitrogen, U.S.A.) for an hour prior to the experiment. The cells were then placed into HPSS with 5 μM GdCl₃ to block endogenous Ca²⁺ entry (added to all superfusions). The M3 AChR-mediated TRPC3 activation was achieved by bath application of carbachol. The mGluR1-mediated TRPC3 activation was achieved by bath application of DHPG.

For Indo-1 experiments, the cells were mounted on a Nikon TMD inverted microscope fitted with an Indo-1 filter set (Nikon Indo-1 filter cube, 485 nm/DM455 nm/405 nm) and illuminated with a mercury lamp. Cells were excited at 350 nm, with dual emission of the field was detected at 410 nm and 480 nm using two photomultiplier tubes, and the ratioed emission was determined in real-time using in-house software. Calibration was performed using a calibration kit and Indo-1 K⁺ salt (Invitrogen, U.S.A.). Ratios were converted to free Ca²⁺ concentrations using the formula from Grynkiewicz et al. (J Biol Chem (1985) 260:3440-3450): [Ca²⁺]=Kd·Q·(R−Rmin)/(Rmax−R), where Kd is the estimated dissociation constant of the Indo-1 and Ca²⁺ with a value of 250 nM, Q is the ratio of Fmin and Fmax at λ2 (480 nm), R represents the fluorescence intensity ratio Fλ1/Fλ2, in which λ1 is at 410 nm.

For Fluo-4 experiments, the cells were mounted on an inverted microscope (DMIL, Leica) with a 20× 0.4NA objective (Leica HCX PL Fluotar), and illuminated with a mercury lamp using a GFP filter (part no. 11504164; excitation 470/40 nm, dichroic 500 nm, emission 525/50 nm) every 5 seconds (Andor iXon+ 885 EMCCD Camera, Ireland) with a gated shutter (Ludl Electronic Products, U.S.A.), controlled via Andor IQ (v1.8.1) software. The images were then analysed using Image J software (NIH, U.S.A.) with individual regions of interest (ROIs) identified for HEK293 cells responding to M3 or mGluR1 agonists with increased Ca²⁺ signal. Change in fluorescence was then presented as a ratio of the basal fluorescence (F0) at the nominal intracellular [Ca²⁺] prior to changing to Ca²⁺-free solution in the bath.

### Results

The initial rise in intracellular [Ca²⁺] in Ca²⁺-free solution following M3 AChR-mediated TRPC3 activation by bath application of carbachol reflected release from Ca²⁺ stores. Carbachol presentation was maintained and extracellular Ca²⁺ restored to nominal levels. This enabled Ca²⁺ entry via the TRPC3 channels (FIG. 6). The resultant peak [Ca²⁺] in HEK293 expressing TRPC3c (575.6±44.2 nM, n=25) was significantly greater than that in TRPC3b expressing cells (182.7±20.8 nM, n=24; p<0.001). The baseline [Ca²⁺] TRPC3c, TRPC3b and untransfected cells prior to CCh treatment were not significantly different (70.1±6.6 nM; 56.2±6.5 nM; 67.4±6.3 nM, respectively, p=0.723, one-way ANOVA). The average peak baseline [Ca²⁺] with return of extracellular Ca²⁺ in untransfected cells was 62.5±5.3 nM, n=6, reflecting an absence of endogenous Ca²⁺ entry under these conditions. CCh-activated Ca²⁺ entry via both TRPC3 channel isoforms was completely blocked by pre-incubation of the cells with genistein, a tyrosine kinase inhibitor (200 μM, typical result for TRPC3c shown in FIG. 6A; TRPC3c, 81.9±19.8 nM, n=5; TRPC3b, 86.7±8.7 nM, n=5). These values did not differ significantly from untransfected control cells with CCh (p=0.341, one-way ANOVA).

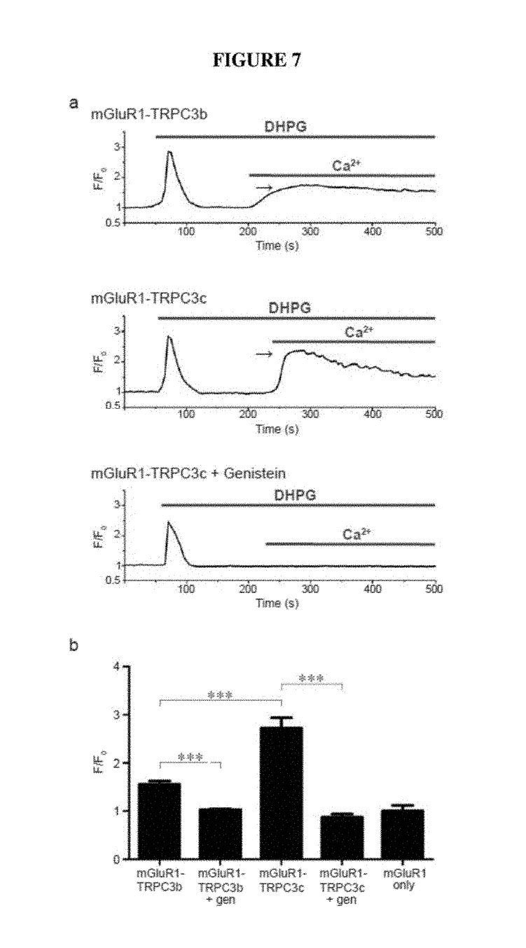

Application of DHPG to HEK293 cells co-expressing TRPC3 and mGluR1 caused an initial rise in Fluo-4 fluorescence in Ca²⁺-free bath solution (expressed as F/F0), reflecting IP₃R-gated Ca²⁺ store activation, as for the carbachol experiments. This was followed, with return of Ca²⁺-containing external solution, by TRPC3-mediated Ca²⁺ entry (FIG. 7). The Fluo-4 fluorescence (average of 5-10 cells per experiment) was significantly greater in cells expressing TRPC3c (TRPC3c, 2.71±0.217, n=12; TRPC3b, 1.56±0.0713, n=10, p<0.001, one-way ANOVA). Pre-application of 200 μM genistein abolished the TRPC3-mediated Ca²⁺ entry (TRPC3c, 0.876±0.0621, n=6; TRPC3b, 1.031±0.0144, n=6, p<0.001, one-way ANOVA).

### Example 5. mGluR1-Activated TRPC3c Current in Cerebellar Purkinje Cells

TRPC3c-mediated current in cerebellar Purkinje cells was then assessed. Parasagittal cerebellar brain slices (400 μm) were prepared using standard techniques (Power and Sah, (2007) J. Physiol. 580:835-57). Mice (C129-SvEv strain, 4-8 weeks old) were anaesthetized with pentobarbital and decapitated. The brain was removed and submerged in an ice cold modified artificial cerebral spinal fluid (ACSF) solution containing 119 mM NaCl, 2.5 mM KCl, 3.3 mM MgCl₂, 0.5 mM CaCl₂, 1.0 mM Na₂PO₄, 26.2 mM NaHCO₃, 11 mM glucose, equilibrated with 95% CO₂, 5% 02. Slices were cut with a VT1200 vibratome (Leica, Germany) and were allowed to recover for at least 1 h at room temperature in a standard ACSF solution containing 119 mM NaCl, 2.5 mM KCl, 1.3 mM MgCl₂, 2.5 mM CaCl₂, 1.0 mM Na₂PO₄, 26.2 mM NaHCO₃, 11 mM glucose, equilibrated with 95% CO₂, 5% O₂.

For recording, slices were transferred to the stage of a Zeiss Examiner D1 microscope and continuously perfused with ACSF heated to 30° C. Whole-cell patch-clamp recordings were made from the soma of Purkinje neurons identified using infrared differential interference contrast videomicroscopy. Patch pipettes (3-5 MΩ) were filled with an internal solution containing 135 mM Cesium methanesulfonate, 8 mM NaCl, 10 mM HEPES, 2 mM Mg₂ATP, 0.3 mM Na₃GTP, 0.1 spermine (pH 7.3 with KOH, osmolarity 290-300 mosmol l⁻¹). Alexa 594 (30 μM; Invitrogen, U.S.A.) was added to the internal solution to visualise the dendritic tree. Whole-cell currents at a holding potential of −70 mV were amplified with an Axopatch 2B amplifier (Molecular Devices, U.S.A.), filtered at 5 kHz and digitized at 20 kHz with a Digidata1440 (Molecular Devices), and controlled using pClamp 10.2 Software (Molecular Devices). Whole-field fluorescence measurements were made with a 40× water immersion objective (NA 1.0; Zeiss, Germany) using a F43 filter block; excitation 545/25 nm; dichroic 570 nm, emission 605/70 nm. Images were acquired with a cooled CCD camera (ProgRes MF-Cool, Jenoptik, Germany) DHPG (50 μM) was applied onto the dendritic field by focal pressure application (in ACSF) through a patch pipette.

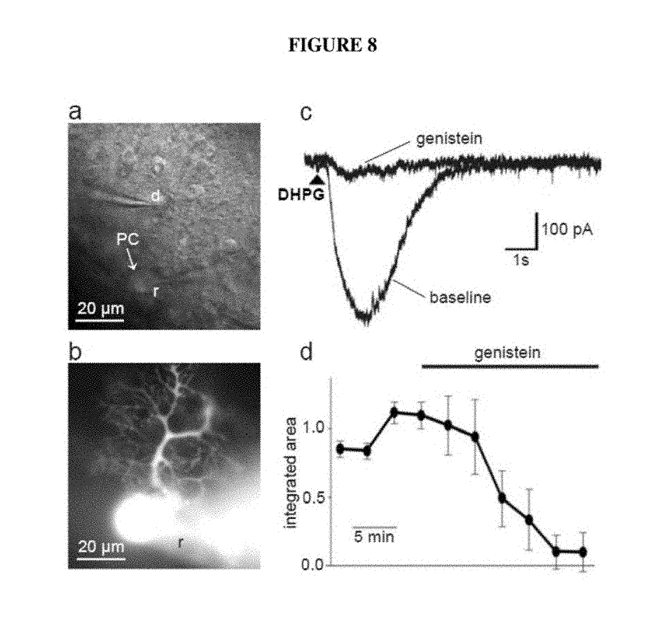

The TRPC3 blocker genistein (100 μM) reduced the DHPG (50 μM)-evoked Purkinje cell inward current by 90±12% (n=3; paired t-test; p=0.036) in mouse cerebellar brain slices (FIG. 8). DHPG-evoked responses could be repeatedly generated at 3 minute intervals prior to addition of genistein to the bath. Genistein produced a block of the current over approximately 10-15 minutes. The sensitivity of the DHPG-activated current to genistein confirms the coupling of the mGluR to the Purkinje cell TRPC3 channels. The peak of the evoked current prior to genistein was −400±55 pA, and −77±50 pA 15 min after genistein (p=0.06). The integrated area of the current (or net charge) was −878±11 pC and −74±105 pC, before and after genistein, respectively.

### Example 6. Effect of Genistein-Mediated Block of TRPC3c Channels on Neuroprotection in a Cerebellar Brain Slice Model of Ischaemic Brain Injury

TRPC3c-mediated brain injury arising from transient oxygen glucose deprivation (OGD) was evaluated in organotypic cerebellar brain slices using the TRPC channel blocker Genistein. The brain slices (400 μm) were prepared as described in Example 5. Mice (C128-SvEv strain, 6-8 weeks old) were anaesthetized with pentobarbital and decapitated. The brain was removed and submerged in ice cold modified artificial cerebral spinal fluid (ACSF) solution containing 4 mM KCl, 5 mM MgCl₂, 1 mM CaCl₂, 26 mM NaHCO₃, 10 mM glucose, 246 mM sucrose equilibrated with 95% CO₂, 5% O₂. Slices were cut with a VT1200 vibratome (Leica, Germany) and were allowed to recover in culture medium (75% Minimum Essential Medium (MEM), 25% heat-inactivated horse serum, 25 mM HEPES, 1 mM glutamine, 27.7 mM glucose) for at least 1 h in a tissue culture incubator (37° C., 5% CO₂ in air). Hurtado de Mendoza et al. (2011) J Vis Exp. (51). (pii):2564).

For OGD-induced neuronal loss, the brain slices were placed in an anaerobic chamber (Coy Laboratory Products, USA; 100% N₂) in OGD medium (75% MEM, 25% Hanks Buffered Salt Solution, 1 mM glutamine) Radley et a. (2012). Neurosci Lett. 506 (1):131-5). Controls included slices placed directly into culture medium in a tissue culture incubator (37° C., 5% CO₂ in air). Slices were exposed to OGD for 0, 15 and 30 minutes, with or without 200 μM genistein (n=2 for each condition). The slices were in organotypic culture overnight, then stained the following day by inclusion of propidium iodide (1 μM for 60 minutes) and then washed three times with culture medium before fixing with paraformaldehyde (4% in 0.1 M phosphate buffer, pH 7.4). The brain slices where then mounted on slides and imaged using a laser scanning microscope with 561 nm excitation.

### Results

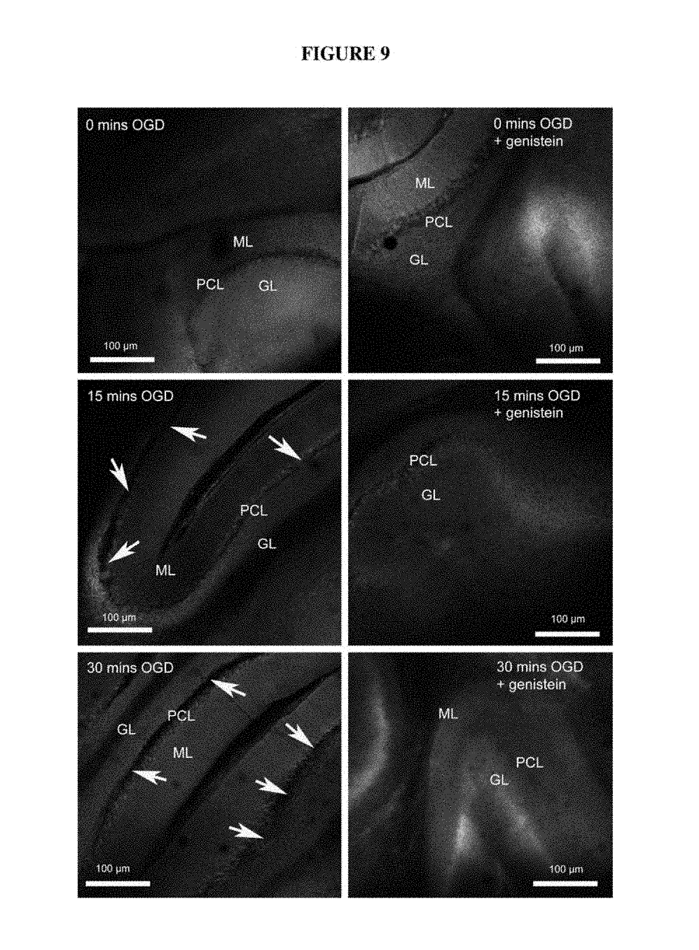

In the control brain slices (OGD only), oedema (tissue swelling) was particularly evident in the Purkinje cell layer (PCL)—arrows. The oedema was more extensive after 30 mins OGD, compared with 15 mins OGD. O mins OGD showed minimal tissue disruption (with or without genistein). Genistein provided protection from the neuronal loss and oedema in the PCL—both at 15 mins and 30 mins (evident from the reduced propodium iodide fluorescence and the lack of swelling (cavity) in the Purkinje cell layer. Examples of a random field from one of the two slices for each treatment are shown in FIG. 9.

1. A method for treating or preventing brain injury associated with excess glutamate release in a subject, comprising administering to the subject a transient receptor potential channel 3 (TRPC3) inhibitor, wherein:
the subject is human;
the subject is experiencing or has experienced an event associated with excess glutamate release; and
the TRPC3 inhibitor is a small molecule that inhibits TRPC3 activity.

2. The method of claim 1, wherein the event associated with excess glutamate release is selected from the group consisting of stroke, epileptic seizure, head trauma, cardiac arrest, severe blood loss, and other ischemic event.

3. The method of claim 1, wherein the event associated with excess glutamate release is stroke.

4. The method of claim 3, wherein the stroke is an ischaemic stroke or a hindbrain stroke.

5. The method of claim 1, further comprising administering an additional therapeutic agent to the subject.

6. The method of claim 5, wherein the additional therapeutic agent is selected from the group consisting of a neuroprotective agent, a thrombolytic agent, insulin, an antiplatelet agent, an anticoagulants and a procoagulant.

7. The method of claim 6, wherein the additional therapeutic agent is a thrombolytic agent and the thrombolytic agent is tissue plasminogen activator.

8. The method of claim 5, wherein the additional therapeutic agent is a thrombolytic agent and wherein the TRPC3 inhibitor and the thrombolytic agent are administered to the subject at the same time or the TRPC3 inhibitor is administered to the subject after the thrombolytic agent is administered to the subject.

9. The method of claim 1, wherein the TRPC3 inhibitor is administered to the subject by a route selected from the group consisting of a parenteral, intravenous, intraarterial, intramuscular, intracranial, intraorbital, nasal, and intraventricular route.

10. The method of claim 1, wherein the TRPC3 inhibitor is a tyrosine kinase inhibitor.

11. The method of claim 1, wherein the TRPC3 inhibitor is selected from the group consisting of genistein (4′, 5, 7-trihydroxyisoflavone or 5, 7-dihydroxy-3-(4-hydroxyphenyl)-4H-1-benzopyran-4-one), PP2 (3-(4-chlorophenyl) 1-(1,1-dimethylethyl)-1H-pyrazolo[3,4-d]pyrimidin-4-amine), 2-aminoethoxydiphenylborane (2-APB), SKF96365, bis(trifluoromethyl)pyrazoles, 4-methyl-4′-[3,5-bis(trifluoromethyl)-1H-pyrazol-1-yl]-1,2,3-thiadiazole-5-carboxanilide (BTP2), ethyl-1-(4-(2,3,3-trichloroacrylamide)phenyl)-5-(trifluoromethyl)-1H-pyrazole-4-carboxylate (Pyr3), norgestimate, erbstatin-analog, herbimycin and lavendustin A.

12. The method of claim 1, wherein the TRPC3 inhibitor is Pyr3 or genistein.
